(19) United States
(12) Patent Application Publication
(10) Pub. No.: US 2025/0099476 A1
(43) Pub. Date: Mar. 27, 2025

(54) SOLID FORMS OF A NUCLEOSIDE ANALOGUE AND USES THEREOF

(71) Applicant: Gilead Sciences, Inc., Foster City, CA (US)

(72) Inventor: Kassibla E. Dempah, San Francisco, CA (US)

(21) Appl. No.: 18/825,549

(22) Filed: Sep. 5, 2024

Related U.S. Application Data
(60) Provisional application No. 63/580,869, filed on Sep. 6, 2023.

(51) Int. Cl. A61K 31/53; C07C 57/145; C07D 487/04

(52) U.S. Cl. CPC A61K 31/53; C07C 57/145; C07D 487/04

(57) ABSTRACT

The disclosure provides solid forms, e.g. salts and crystalline forms thereof, of the compound of Formula I and uses thereof. Also provided are the methods of making the solid forms, as well as pharmaceutical formulations and kits comprising the solid forms.

### CROSS REFERENCE TO RELATED APPLICATIONS

This application claims the benefit of U.S. Provisional Application No. 63/580,869 filed on Sep. 6, 2023, the entire content of which is hereby incorporated by reference in its entirety.

### TECHNICAL FIELD

The present disclosure relates to solid forms of ((2R,3S,4R,5R)-5-(4-aminopyrrolo[2,1-f][1,2,4]triazin-7-yl)-5-cyano-3,4-dihydroxytetrahydrofuran-2-yl)methyl isopropyl carbonate, and their use in the treatment of a viral infections. The present disclosure also relates to pharmaceutical compositions comprising the solid forms disclosed herein, and methods of treating or preventing viral infections.

### BACKGROUND

There is an ongoing need for antiviral agents and methods for treating viral infections, for example paramyxoviridae, pneumoviridae, picornaviridae, flaviviridae, filoviridae, arenaviridae, orthomyxovirus, poxvirus, and coronaviridae infections. There is also a constant need to develop methods for preparation and purification of the antiviral agents, as well as prepare improved pharmaceutical formulations of the same. The solid forms disclosed herein help meet these and other needs.

### SUMMARY

In present disclosure solid forms (for example salts and crystalline forms thereof) of a compound of Formula I:

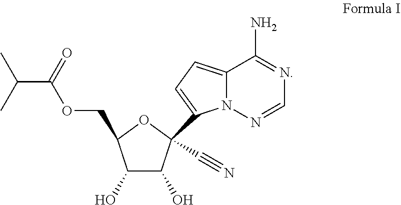 [SMILES: CC(C)C(=O)OC[C@H]1O[C@@](C#N)(c2ccc3c(N)ncnn23)[C@H](O)[C@@H]1O]

In one aspect the disclosure provides a maleate salt of a compound of Formula I:

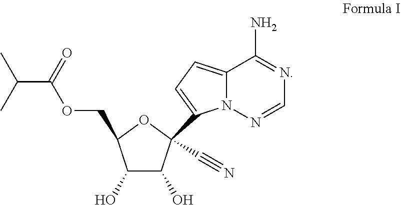 [SMILES: CC(C)C(=O)OC[C@H]1O[C@@](C#N)(c2ccc3c(N)ncnn23)[C@H](O)[C@@H]1O]

In another aspect the disclosure provides a crystalline form of the maleate salt of a compound of Formula I.

In another aspect, the present disclosure provides an oxalate salt of the compound of Formula I.

In another aspect, the present disclosure provides a crystalline form of the oxalate salt of the compound of Formula I.

In another aspect, the present disclosure provides a methanesulfonate salt of the compound of Formula I.

In another aspect, the present disclosure provides a crystalline form of the methanesulfonate salt of the compound of Formula I.

In another aspect, the present disclosure provides a ethanesulfonate salt of the compound of Formula I.

In another aspect, the present disclosure provides a crystalline form of the ethanesulfonate salt of the compound of Formula I.

The present disclosure further provides methods of making the solid forms disclosed herein.

The present disclosure further provides a pharmaceutical composition comprising the solid forms disclosed herein and a pharmaceutically acceptable excipient.

The present disclosure further provides a kit comprising the solid forms disclosed herein and a pharmaceutically acceptable excipient.

The present disclosure further provides a method of treating or preventing a viral infection in a human in need thereof, wherein the method comprises administering to the human a solid form or a pharmaceutical composition disclosed herein.

The present disclosure further provides a method for manufacturing a medicament for treating or preventing a viral infection in a human in need thereof, characterized in that a solid form or pharmaceutical composition disclosed herein is used.

The present application further provides use of a solid forms or pharmaceutical composition disclosed herein for the manufacture of a medicament for the treatment or prevention of a viral infection in a human in need thereof.

The present application further provides a solid form or pharmaceutical composition disclosed herein for use in treatment or prevention of a viral infection in a human in need thereof.

### BRIEF DESCRIPTION OF THE DRAWINGS

FIG. 1. Shows the XRPD pattern of the compound of Formula I maleate

FIG. 2. Shows the DSC thermogram of the compound of Formula I maleate.

FIG. 3. Shows the TGA thermogram of the compound of Formula I maleate.

FIG. 4. Shows the XRPD pattern of the compound of Formula I oxalate.

FIG. 5. Shows the DSC thermogram the compound of Formula I oxalate.

FIG. 6. Shows the TGA thermogram of the compound of Formula I oxalate.

FIG. 7. Shows the XRPD pattern of the compound of Formula I methanesulfonate Material A.

FIG. 8. Shows the XRPD pattern of the compound of Formula I methanesulfonate Material B.

FIG. 9. Shows the TGA thermogram of the compound of Formula I methanesulfonate Material B.

FIG. 10. Shows the XRPD pattern of the compound of Formula I ethanesulfonate.

### DETAILED DESCRIPTION

The present disclosure relates to new solid forms of ((2R,3S,4R,5R)-5-(4-aminopyrrolo[2,1-f][1,2,4]triazin-7-yl)-5-cyano-3,4-dihydroxytetrahydrofuran-2-yl)methyl isopropyl carbonate (i.e. the compound of Formula I, see below). One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC).

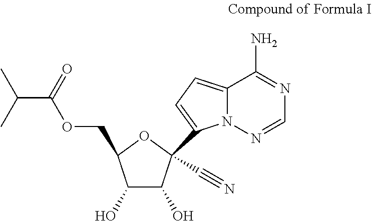 [SMILES: CC(C)C(=O)OC[C@H]1O[C@@](C#N)(c2ccc3c(N)ncnn23)[C@H](O)[C@@H]1O]

The solid forms of the invention include salt forms (both amorphous and crystalline) as well as cocrystal forms of the compound of Formula I. As used herein, “solid form” generally refers to a solid chemical substance that can be amorphous or crystalline. In some embodiments, the solid form of the invention is a salt of compound of Formula I which can be amorphous or crystalline. In further embodiments, the solid form can be a cocrystal of compound of Formula I, in which compound of Formula I has formed a crystalline solid together with a coformer molecule. In some embodiments, the solid form is a solvate (e.g. a hydrate). Both crystalline salts and cocrystals of compound of Formula I can exist in different crystalline forms (i.e., have different polymorphic or pseudopolymorphic forms).

As used herein, the term “cocrystal” refers to a compound (such as compound of Formula I) crystallized together with one or more coformer molecules (e.g., molecules other than the compound). Depending on the chemical nature and proportion of coformers present in the cocrystal, different physical properties related to, for example, dissolution and solubility may be observed compared with solid forms of the compound by itself or salts thereof. In some instances, the coformer molecule may be a protic acid, and whether the protic acid forms a salt or a cocrystal will often depend on the relative pKa's of the compound and coformer. See, e.g., Regulatory Classification of Pharmaceutical Co-Crystals: Guidance for Industry, revised August 2016, published by the U.S. Dept. of Health and Human Services, FDA, Center for Drug Evaluation and Research (CDER).

As used herein, “crystalline form” is meant to refer to a certain lattice configuration of a crystalline substance (e.g., a salt or a cocrystal). Different crystalline forms of the same substance typically have different crystalline lattices (e.g., unit cells) which are attributed to different physical properties that are characteristic of each of the crystalline forms. In some instances, different lattice configurations have different water or solvent content.

According to the present invention, a solid forms of compound of Formula I can be useful in the synthesis and/or purification of the compound of Formula I. For example, a solid form of compound of Formula I can be an intermediate in the synthesis of the compound 1 of Formula I. In addition, different solid forms of compound of Formula I may have different properties with respect to bioavailability, stability, purity, and/or manufacturability for medical or pharmaceutical uses. Variations in the solid form of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, some solid (e.g. crystalline forms) may provide desired or suitable hygroscopicity, particle size controls, dissolution rate, solubility, purity, physical and chemical stability, manufacturability, yield, and/or process control. Thus, the solid forms of compound of Formula I may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different solid forms of compound of Formula I which may exhibit one or more of the favorable characteristics described above. The processes for the preparation of the solid forms described herein and characterization of these solid forms are described in detail below.

In some embodiments, the solid forms described herein are purified or substantially isolated. By “substantially isolated” is meant that the crystalline form is at least partially or substantially separated from the environment in which it was formed or detected. Partial separation can include, for example, a composition enriched in the crystalline form of the disclosure. Substantial separation can include compositions containing at least about 50%, at least about 60%, at least about 70%, at least about 80%, at least about 90%, at least about 95%, at least about 97%, or at least about 99% by weight of the crystalline form of the disclosure. In some embodiments, the crystalline form of the disclosure can be prepared with a purity of about 75% or more, 80% or more, 85% or more, 90% or more, 95% or more, 98% or more, or 99% or more.

The different crystalline forms can be identified by solid state characterization methods such as by X-ray powder diffraction (XRPD). Other characterization methods such as differential scanning calorimetry (DSC) and thermogravimetric analysis (TGA) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2θ values. Thus, peak assignments, such as those reported herein, can vary by plus or minus about 0.2° (2θ), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The present invention provides crystalline forms of the compound of Formula I or a pharmaceutically acceptable salt thereof. In some embodiments, the crystalline form may be substantially anhydrous. In some embodiments, the crystalline form may be hydrated or solvated.

### Compound of Formula I, Maleate Slat and Crystalline Forms Thereof

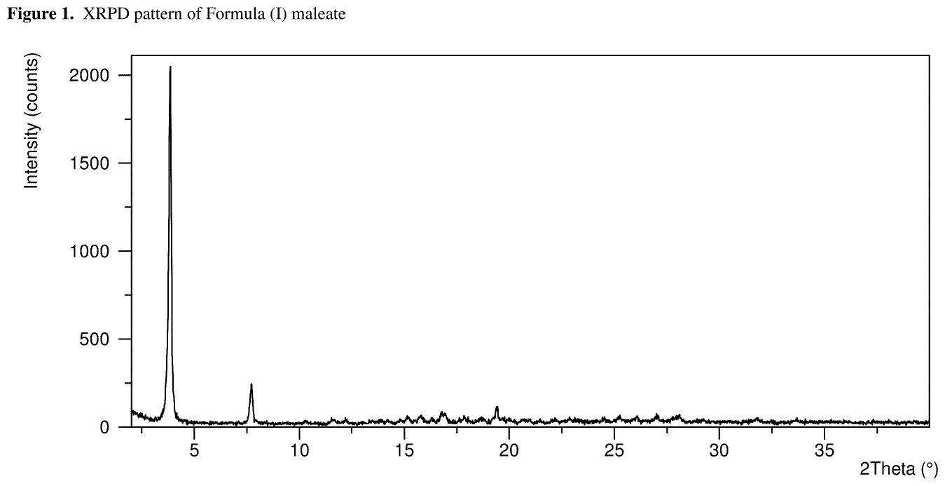

In some embodiments, the present disclosure provides a maleate salt of the compound of Formula I (“the compound of Formula I, maleate salt). In some embodiments, the compound of Formula I, maleate salt is crystalline. In some embodiments, the compound of Formula I, maleate salt is a crystalline form having an XRPD profile substantially as shown in FIG. 1. In some embodiments, compound of Formula I, maleate salt is a crystalline form having an XRPD pattern displaying at least two, at least three, at least four, at least five, or at least six of the 2θ-reflections as the XRPD pattern substantially as shown in FIG. 1.

In some embodiments, the compound of Formula I, maleate salt crystalline form is characterized by an XRPD pattern comprising 2θ-reflections (±0.2 degrees 2θ) at about 3.9°, 7.7°, and 19.4°. In some embodiments, the compound of Formula I, maleate salt crystalline form has an XRPD pattern comprising 2θ-reflections (±0.2 degrees 2θ) at 3.9°, 7.7°, and 19.4°, and one, two or three of the 2θ-reflections (±0.2 degrees 2θ) at 2.2°, 15.2°, and 27.0°. In some embodiments, the compound of Formula I, maleate salt crystalline form has an XRPD pattern comprising 2θ-reflections (±0.2 degrees 2θ) at 3.9°, 7.7°, and 19.4°, and one or two of the 2θ-reflections (±0.2 degrees 2θ) at 2.2°, 15.2°, and 27.0°. In some embodiments, the compound of Formula I, maleate salt crystalline form has an XRPD pattern comprising 2θ-reflections (±0.2 degrees 2θ) at 3.9°, 7.7°, and 19.4°, and one of the 2θ-reflections (±0.2 degrees 2θ) at 2.2°, 15.2°, and 27.0°. In some embodiments, the compound of Formula I, maleate salt crystalline form has an XRPD pattern comprising 2θ-reflections (±0.2 degrees 2θ) at 3.9°, 7.7°, and 19.4°, and two of the 2θ-reflections (±0.2 degrees 2θ) at 2.2°, 15.2°, and 27.0°. In some embodiments, the compound of Formula I, maleate salt crystalline form has an XRPD pattern comprising 2θ-reflections (±0.2 degrees 2θ) at 2.2°, 3.9°, 7.7°, 15.2°, 19.4° and 27.0°. In some embodiments, the compound of Formula I, maleate salt crystalline form has an XRPD pattern comprising any three 2θ-reflections (±0.2 degrees 2θ) selected from the group consisting of 2.2°, 3.9°, 7.7°, 15.2°, 19.4° and 27.0°.

In some embodiments, the compound of Formula I, maleate salt crystalline form has an XRPD pattern comprising 2θ-reflections (±0.2 degrees 2θ) at 2.2°, 3.9°, 7.7°, 15.2°, 19.4° and 27.0°, and one, two or three of the 2θ-reflections (±0.2 degrees 2θ) at 11.6°, 12.2°, and 28.1°. In some embodiments, the compound of Formula I, maleate salt crystalline form has an XRPD pattern comprising 2θ-reflections (±0.2 degrees 2θ) at 2.2°, 3.9°, 7.7°, 15.2°, 19.4° and 27.0°, and one or two of the 2θ-reflections (±0.2 degrees 2θ) at 11.6°, 12.2°, and 28.1°. In some embodiments, the compound of Formula I, maleate salt crystalline form has an XRPD pattern comprising 2θ-reflections (±0.2 degrees 2θ) at 2.2°, 3.9°, 7.7°, 15.2°, 19.4° and 27.0°, and one of the 2θ-reflections (±0.2 degrees 2θ) at 11.6°, 12.2°, and 28.1°. In some embodiments, the compound of Formula I, maleate salt crystalline form has an XRPD pattern comprising 20˜ reflections (±0.2 degrees 2θ) at 2.2°, 3.9°, 7.7°, 15.2°, 19.4° and 27.0°, and two of the 2θ-reflections (±0.2 degrees 2θ) at 11.6°, 12.2°, and 28.1°. In some embodiments, the compound of Formula I, maleate salt crystalline form has an XRPD pattern comprising 2θ-reflections (±0.2 degrees 2θ) at 2.2°, 3.9°, 7.7°, 11.6°, 12.2°, 15.2°, 19.4° 27.0°, and 28.1°. In some embodiments, the compound of Formula I, maleate salt crystalline form has an XRPD pattern comprising any three 2θ-reflections (±0.2 degrees 2θ) selected from the group consisting of 2.2°, 3.9°, 7.7°, 11.6°, 12.2°, 15.2°, 19.4°, 27.0°, and 28.1°.

In some embodiments, the compound of Formula I, maleate salt crystalline form has a XRPD pattern comprising peaks at:

| Pos. [°2Th.] | Rel. Int. [%] |
|---|---|
| 2.2 | 3 |
| 3.9 | 100 |
| 7.7 | 10 |
| 11.6 | 1 |
| 12.2 | 1 |
| 15.2 | 2 |
| 15.8 | 2 |
| 16.8 | 2 |
| 17.9 | 1 |
| 18.7 | 1 |
| 19.4 | 4 |
| 22.9 | 1 |
| 25.2 | 1 |
| 26.0 | 1 |
| 27.0 | 2 |
| 28.1 | 2 |
| 31.8 | 1 |

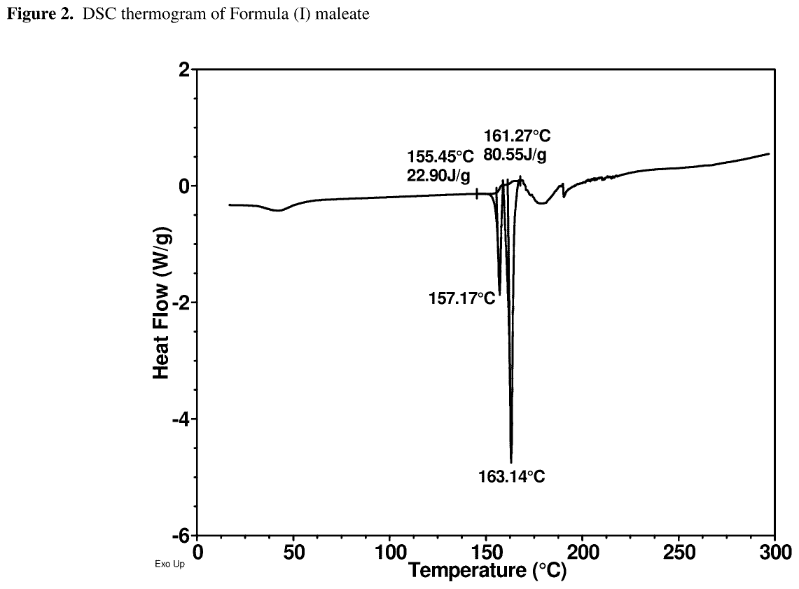

In some embodiments, the compound of Formula I, maleate salt crystalline form is characterized by a DSC thermogram substantially as shown in FIG. 2. In some embodiments, the compound of Formula I, maleate salt, crystalline form is characterized by a DSC thermogram comprising two endothermic transitions at about 155° C. and 161° C. In some embodiments, the compound of Formula I, maleate salt, crystalline form is characterized by a DSC thermogram comprising an endothermic transitions at about 155° C. and/or 161° C.

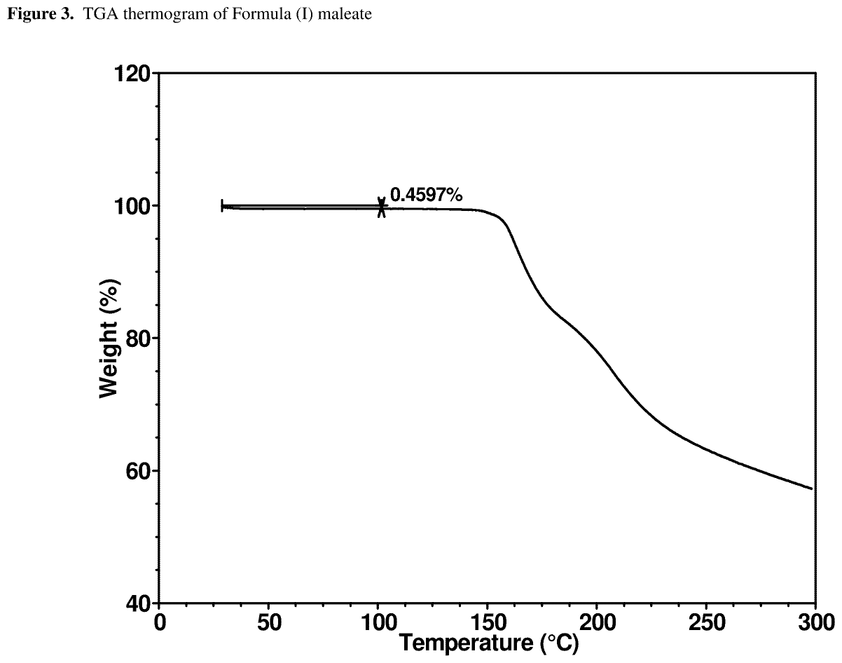

In some embodiments, the compound of Formula I, maleate salt, crystalline form is characterized by a TGA curve substantially as shown in FIG. 3.

In some embodiments, the compound of Formula I, maleate salt crystalline form is a solvate.

In some embodiments, the compound of Formula I, maleate salt crystalline form is not a solvate.

### Compound of Formula I, Oxalate Slat and Crystalline Forms Thereof

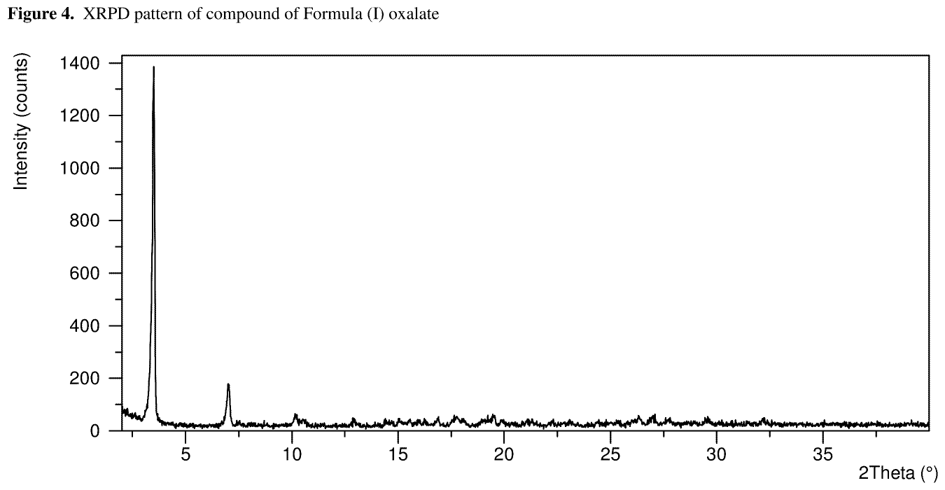

In some embodiments, the present disclosure provides a oxalate salt of the compound of Formula I (i.e. compound of Formula I, oxalate salt). In some embodiments, the compound of Formula I, oxalate salt is crystalline. In some embodiments, the compound of Formula I, oxalate salt is a crystalline form having an XRPD profile substantially as shown in FIG. 4. In some embodiments, compound of Formula I, oxalate salt is a crystalline form having an XRPD pattern displaying at least two, at least three, at least four, at least five, or at least six of the 2θ-reflections as the XRPD pattern substantially as shown in FIG. 4.

In some embodiments, the compound of Formula I, oxalate salt crystalline form is characterized by an XRPD pattern comprising 2θ-reflections (±0.2 degrees 2θ) at about 3.5°, 7.0°, and 17.7°. In some embodiments, compound of Formula I, oxalate salt crystalline form has an XRPD pattern comprising 2θ-reflections (±0.2 degrees 2θ) at 3.5°, 7.0°, and 17.7°, and one, two or three of the 2θ-reflections (±0.2 degrees 2θ) at 10.2°, 19.5°, and 27.0°. In some embodiments, the compound of Formula I, oxalate salt crystalline form has an XRPD pattern comprising 2θ-reflections (±0.2 degrees 2θ) at 3.5°, 7.0°, and 17.7°, and one or two of the 2θ-reflections (±0.2 degrees 2θ) at 10.2°, 19.5°, and 27.0°. In some embodiments, the compound of Formula I, oxalate salt crystalline form has an XRPD pattern comprising 2θ-reflections (±0.2 degrees 2θ) at 3.5°. 7.0°, and 17.7°, and one of the 2θ-reflections (±0.2 degrees 2θ) at 10.2°, 19.5°, and 27.0°. In some embodiments, the compound of Formula I, oxalate salt crystalline form has an XRPD pattern comprising 2θ-reflections (±0.2 degrees 2θ) at 3.5°, 7.0°, and 17.7°, and two of the 2θ-reflections (±0.2 degrees 2θ) at 10.2°, 19.5°, and 27.0°. In some embodiments, the compound of Formula I, oxalate salt crystalline form has an XRPD pattern comprising 2θ-reflections (±0.2 degrees 2θ) at 3.5°, 7.0°, 10.2°, 17.7°, 19.5°, and 27.0°. In some embodiments, the compound of Formula I, oxalate salt crystalline form has an XRPD pattern comprising any three 2θ-reflections (±0.2 degrees 2θ) selected from the group consisting of 3.5°, 7.0°, 10.2°, 17.7°, 19.5°, and 27.0°.

In some embodiments, the compound of Formula I, oxalate salt crystalline form has an XRPD pattern comprising 2θ-reflections (±0.2 degrees 2θ) at 3.5°, 7.0°, 10.2°, 17.7°, 19.5°, and 27.0°, and one, two or three of the 2θ-reflections (±0.2 degrees 2θ) at 12.9°, 21.2°, and 29.5°. In some embodiments, the compound of Formula I, oxalate salt crystalline form has an XRPD pattern comprising 2θ-reflections (±0.2 degrees 2θ) at 3.5°, 7.0°, 10.2°, 17.7°, 19.5°, and 27.0°, and one or two of the 2θ-reflections (±0.2 degrees 2θ) at 12.9°, 21.2°, and 29.5°. In some embodiments, the compound of Formula I, oxalate salt crystalline form has an XRPD pattern comprising 2θ-reflections (±0.2 degrees 2θ) at 3.5°, 7.0°, 10.2°, 17.7°, 19.5°, and 27.0°, and one of the 2θ-reflections (±0.2 degrees 2θ) at 12.9°, 21.2°, and 29.5°. In some embodiments, the compound of Formula I, oxalate salt crystalline form has an XRPD pattern comprising 2θ-reflections (±0.2 degrees 2θ) at 3.5°, 7.0°, 10.2°, 17.7°, 19.5°, and 27.0°, and two of the 2θ-reflections (±0.2 degrees 2θ) at 12.9°, 21.2°, and 29,5°. In some embodiments, the compound of Formula I, oxalate salt crystalline form has an XRPD pattern comprising 2θ-reflections (±0.2 degrees 2θ) at 3.5°, 7.0°, 10.2°, 12.9°, 17.7°, 19.5°, 21.2°, 27.0°, and 29.5°. In some embodiments, the compound of Formula I, oxalate salt crystalline form has an XRPD pattern comprising any three 2θ-reflections (±0.2 degrees 2θ) selected from the group consisting of 3.5°, 7.0°, 10.2°, 12.9°, 17.7°, 19.5°, 21.2°, 27.0°, and 29.5°.

In some embodiments, the compound of Formula I, oxalate salt crystalline form has a XRPD pattern comprising peaks at:

| Pos. [°2Th.] | Rel. Int. [%] |
|---|---|
| 3.5 | 100 |
| 7.0 | 12 |
| 10.2 | 3 |
| 12.9 | 1 |
| 16.8 | 1 |
| 17.7 | 2 |
| 19.5 | 2 |
| 21.2 | 1 |
| 26.5 | 1 |
| 27.0 | 2 |
| 27.7 | 1 |
| 29.5 | 1 |
| 32.2 | 1 |

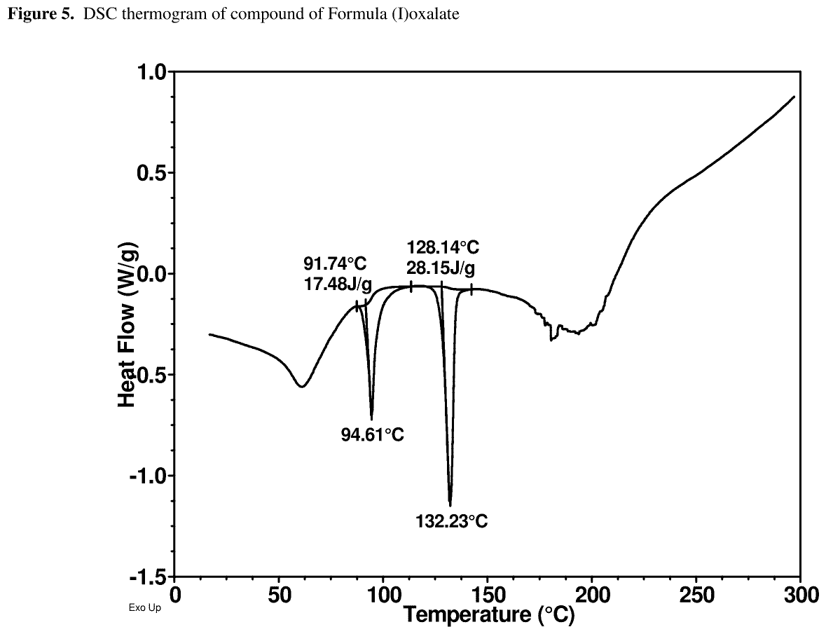

In some embodiments, compound of Formula I, oxalate salt crystalline form is characterized by a DSC thermogram substantially as shown in FIG. 5. In some embodiments, the compound of Formula I, oxalate salt crystalline form is characterized by a DSC thermogram having two endothermic events at about 92° C. and at about 128° C. In some embodiments, the compound of Formula 1, oxalate salt crystalline form is characterized by a DSC thermogram having two endothermic events at about 92° C. and/or about 128° C.

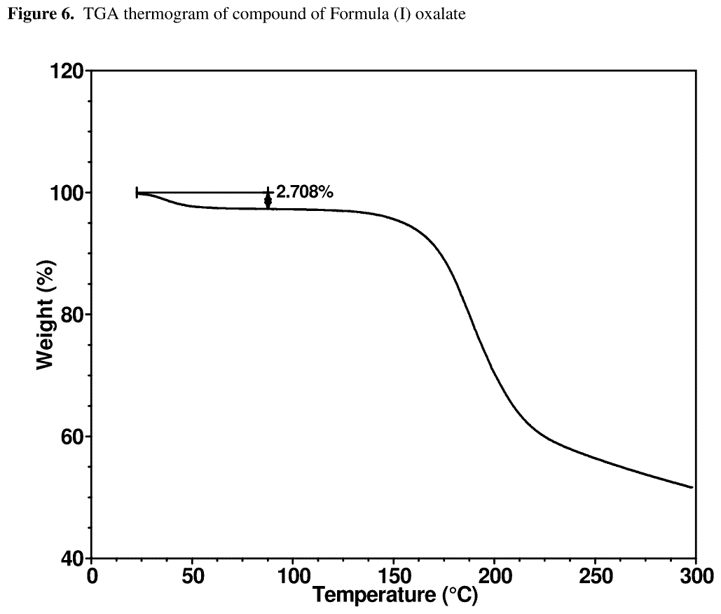

In some embodiments, the compound of Formula I, oxalate salt crystalline form is characterized by a TGA curve substantially as shown in FIG. 6.

In some embodiments, the compound of Formula I, oxalate salt crystalline form is a solvate.

In some embodiments, the compound of Formula I, oxalate salt crystalline form is not a solvate.

### Compound of Formula I, Methanesulfonate Slat and Crystalline Forms Thereof

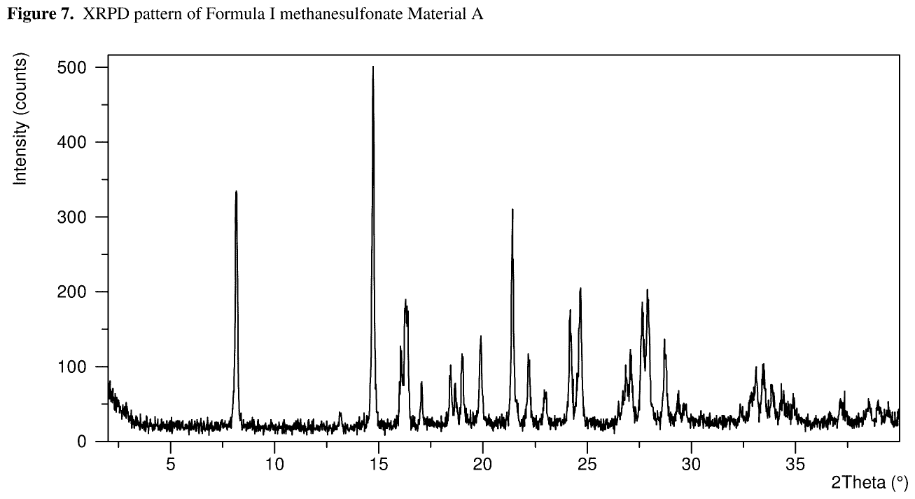

In some embodiments, the present disclosure provides a methanesulfonate salt of the compound of Formula I (i.e. compound of Formula I, methanesulfonate salt). In some embodiments, the compound of Formula I, methanesulfonate salt is crystalline. In some embodiments, the compound of Formula I, methanesulfonate salt is a crystalline form having an XRPD profile substantially as shown in FIG. 7. In some embodiments, compound of Formula I, methanesulfonate salt is a crystalline form having an XRPD pattern displaying at least two, at least three, at least four, at least five, or at least six of the 2θ-reflections as the XRPD pattern substantially as shown in FIG. 7.

In some embodiments, the compound of Formula I, methanesulfonate salt crystalline form is characterized by an XRPD pattern comprising 2θ-reflections (±0.2 degrees 2θ) at about 8.1°, 14.7°, and 21.4°. In some embodiments, compound of Formula I, methanesulfonate salt crystalline form has an XRPD pattern comprising 2θ-reflections (±0.2 degrees 2θ) at 8.1°, 14.7°, and 21.4°, and one, two or three of the 2θ-reflections (±0.2 degrees 2θ) at 16.3°, 19.9°, and 24.7°. In some embodiments, the compound of Formula I, methanesulfonate salt crystalline form has an XRPD pattern comprising 2θ-reflections (±0.2 degrees 2θ) at 8.1°, 14.7°, and 21.4°, and one or two of the 2θ-reflections (±0.2 degrees 2θ) at 16.3°, 19.9°, and 24.7°. In some embodiments, the compound of Formula I, methanesulfonate salt crystalline form has an XRPD pattern comprising 2θ-reflections (±0.2 degrees 2θ) at 8.1°, 14.7°, and 21.4°, and one of the 2θ-reflections (±0.2 degrees 2θ) at 16.3°, 19.9°, and 24.7°. In some embodiments, the compound of Formula I, methanesulfonate salt crystalline form has an XRPD pattern comprising 2θ-reflections (±0.2 degrees 2θ) at 8.1°, 14.7°, and 21.4°, and two of the 2θ-reflections (±0.2 degrees 2θ) at 16.3°, 19.9°, and 24.7°. In some embodiments, the compound of Formula I, methanesulfonate salt crystalline form has an XRPD pattern comprising 2θ-reflections (±0.2 degrees 2θ) at 8.1°, 14.7°, 16.3°, 19.9°, 21,4°, and 24.7°. In some embodiments, the compound of Formula I, methanesulfonate salt crystalline form has an XRPD pattern comprising any three 2θ-reflections (±0.2 degrees 2θ) selected from the group consisting of 8.1°, 14.7°,16.39, 19.9°, 21.4°, and 24.7°,

In some embodiments, the compound of Formula I, methanesulfonate salt crystalline form has an XRPD pattern comprising 2θ-reflections (±0.2 degrees 2θ) at 8.1°, 14.7°, 16.3°. 19.9°, 21.4°, and 24.7°, and one, two or three of the 2θ-reflections (±0.2 degrees 2θ) at 19.0°, 24.2°, and 27.9°. In some embodiments, the compound of Formula I, methanesulfonate salt crystalline form has an XRPD pattern comprising 2θ-reflections (±0.2 degrees 2θ) at 8.1°, 14.7°,16.3°, 19.9°, 21.4°, and 24.7°, and one or two of the 2θ-reflections (±0.2 degrees 2θ) at 19.0°, 24.2°, and 27.9°. In some embodiments, the compound of Formula I, methanesulfonate salt crystalline form has an XRPD pattern comprising 2θ-reflections (±0.2 degrees 2θ) at 8.1°, 14.7°,16.3°, 19.9°, 21,4°, and 24.7°, and one of the 2θ-reflections (±0.2 degrees 2θ) at 19.0°, 24.2°, and 27.9°. In some embodiments, the compound of Formula I, methanesulfonate salt crystalline form has an XRPD pattern comprising 2θ-reflections (±0.2 degrees 2θ) at 8.1°, 14.7°,16.3°, 19.9°, 21.4°, and 24.7°, and two of the 2θ-reflections (±0.2 degrees 2θ) at 19.0°, 24.2°, and 27.9°. In some embodiments, the compound of Formula I, methanesulfonate salt crystalline form has an XRPD pattern comprising 2θ-reflections (±0.2 degrees 2θ) at 8.1°, 14.7°,16.3°, 19.0°, 19.9°, 21.4°, 24.2°, 24.7°, and 27.9°. In some embodiments, the compound of Formula I, methanesulfonate salt crystalline form has an XRPD pattern comprising any three 2θ-reflections (±0.2 degrees 2θ) selected from the group consisting of 8.1°, 14.7°, 16.3°, 19.0°, 19.9°, 21.4°, 24.2°, 24.7°, and 27.9°.

In some embodiments, the compound of Formula I, methanesulfonate salt crystalline form has a XRPD pattern comprising peaks at:

| Pos. [°2Th.] | Rel. Int. [%] |
|---|---|
| 8.1 | 69 |
| 13.1 | 3 |
| 14.7 | 100 |
| 16.3 | 35 |
| 17.0 | 12 |
| 18.4 | 14 |
| 18.7 | 11 |
| 19.0 | 20 |
| 19.9 | 26 |
| 21.4 | 59 |
| 22.2 | 21 |
| 23.0 | 9 |
| 24.2 | 31 |
| 24.7 | 39 |
| 27.1 | 19 |
| 27.6 | 32 |
| 27.9 | 37 |
| 28.7 | 21 |
| 29.4 | 7 |
| 33.1 | 14 |
| 33.5 | 15 |
| 33.9 | 11 |
| 34.3 | 10 |
| 34.9 | 8 |
| 37.2 | 5 |
| 38.5 | 5 |
| 39.0 | 6 |
| 39.3 | 6 |

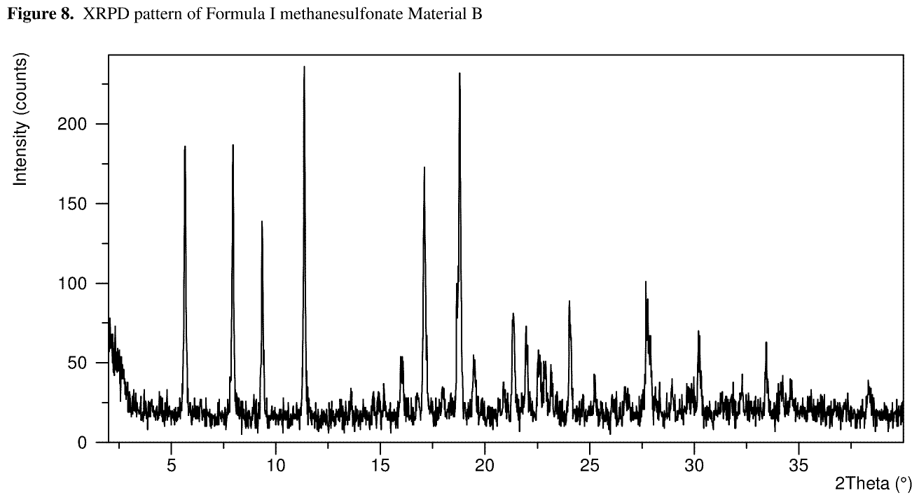

In some embodiments, the compound of Formula I, methanesulfonate salt is a crystalline form having an XRPD profile substantially as shown in FIG. 8. In some embodiments, compound of Formula I, methanesulfonate salt is a crystalline form having an XRPD pattern displaying at least two, at least three, at least four, at least five, or at least six of the 2q-reflections as the XRPD pattern substantially as shown in FIG. 8.

In some embodiments, the compound of Formula I, methanesulfonate salt crystalline form is characterized by an XRPD pattern comprising 2θ-reflections (±0.2 degrees 2θ) at about 5.7°, 11.4°, and 18.8°. In some embodiments, compound of Formula I, methanesulfonate salt crystalline form has an XRPD pattern comprising 2θ-reflections (±0.2 degrees 2θ) at 5.7°, 11.4°, and 18.8°, and one, two or three of the 2θ-reflections (±0.2 degrees 2θ) at 7.9°, 17.1°, and 27.7°. In some embodiments, the compound of Formula I, methanesulfonate salt crystalline form has an XRPD pattern comprising 2θ-reflections (±0.2 degrees 2θ) at 5.7°, 11.4°, and 18.8°, and one or two of the 2θ-reflections (±0.2 degrees 2θ) at 7.9°, 17.1°, and 27.7°. In some embodiments, the compound of Formula I, methanesulfonate salt crystalline form has an XRPD pattern comprising 2θ-reflections (±0.2 degrees 2θ) at 5.7°, 11.4°, and 18.8°, and one of the 2θ-reflections (±0.2 degrees 2θ) at 7.9°, 17.1°, and 27.7°. In some embodiments, the compound of Formula I, methanesulfonate salt crystalline form has an XRPD pattern comprising 2θ-reflections (±0.2 degrees 2θ) at 5.7°, 11.4°, and 18.8°, and two of the 2θ-reflections (±0.2 degrees 2θ) at 7.9°, 17.1°, and 27.7°. In some embodiments, the compound of Formula I, methanesulfonate salt crystalline form has an XRPD pattern comprising 2θ-reflections (±0.2 degrees 2θ) at 5.7°, 7.9°, 11.4°, 17.1°, 18.8°, and 27.7°. In some embodiments, the compound of Formula I, methanesulfonate salt crystalline form has an XRPD pattern comprising any three 2θ-reflections (±0.2 degrees 2θ) selected from the group consisting of 5.7°, 7.9°, 11.4°, 17.1°, 18.8°, and 27.7°.

In some embodiments, the compound of Formula I, methanesulfonate salt crystalline form has an XRPD pattern comprising 2θ-reflections (±0.2 degrees 2θ) at 5.7°, 7.9°, 11.4°, 17.1°, 18.8°, and 27.7°, and one, two or three of the 2θ-reflections (±0.2 degrees 2θ) at 9.4°, 21.3°, and 24.1°. In some embodiments, the compound of Formula I, methanesulfonate salt crystalline form has an XRPD pattern comprising 2θ-reflections (±0.2 degrees 2θ) at 5.7°, 7.9°, 11.4°, 17.1°, 18.8°, and 27.7°, and one or two of the 2θ-reflections (±0.2 degrees 2θ) at 9.4°, 21.3°, and 24.1°. In some embodiments, the compound of Formula I, methanesulfonate salt crystalline form has an XRPD pattern comprising 2θ-reflections (±0.2 degrees 2θ) at 5.7°, 7.9°, 11.4°, 17.1°, 18.8°, and 27.7°, and one of the 2θ-reflections (±0.2 degrees 2θ) at 9.4°, 21.3°, and 24.1°. In some embodiments, the compound of Formula I, methanesulfonate salt crystalline form has an XRPD pattern comprising 2θ-reflections (±0.2 degrees 2θ) at 5.7°, 7.9°, 11.4°, 17.1°, 18.8°, and 27.7°, and two of the 2θ-reflections (±0.2 degrees 2θ) at 9.4°, 21.3°, and 24.1°. In some embodiments, the compound of Formula I, methanesulfonate salt crystalline form has an XRPD pattern comprising 2θ-reflections (±0.2 degrees 2θ) at 5.7°, 7.9°, 9.4°, 11.4°, 17.1°, 18.8°, 21.3°, 24.1°, 27.7°. In some embodiments, the compound of Formula I, methanesulfonate salt crystalline form has an XRPD pattern comprising any three 2θ-reflections (±0.2 degrees 2θ) selected from the group consisting of 5.7°, 7.9°, 9.4°, 11.4°, 17.1°, 18.8°, 21.3°, 24.1°, and 27.7°.

In some embodiments, the compound of Formula I, methanesulfonate salt crystalline form has a XRPD pattern comprising peaks at:

| Pos. [°2Th.] | Rel. Int. [%] |
|---|---|
| 5.7 | 77 |
| 7.9 | 75 |
| 9.4 | 55 |
| 11.4 | 99 |
| 13.6 | 16 |
| 15.2 | 17 |
| 16.0 | 13 |
| 17.1 | 70 |
| 18.8 | 100 |
| 19.5 | 15 |
| 21.3 | 29 |
| 22.0 | 22 |
| 22.6 | 15 |
| 23.2 | 10 |
| 24.1 | 27 |
| 27.7 | 29 |
| 30.2 | 20 |
| 32.3 | 10 |
| 33.4 | 15 |
| 34.2 | 10 |
| 34.7 | 10 |
| 38.4 | 7 |

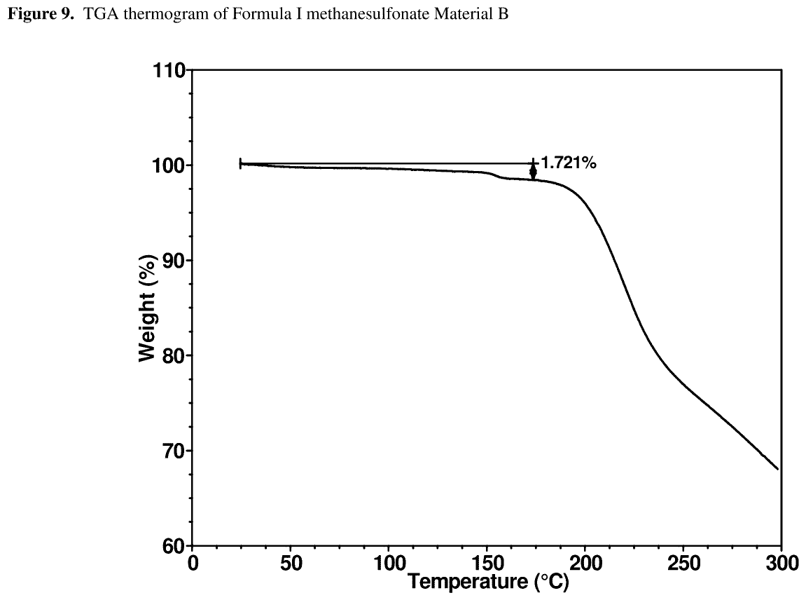

In some embodiments, the compound of Formula I, methanesulfonate salt crystalline form is characterized by a TGA curve substantially as shown in FIG. 9.

In some embodiments, the compound of Formula I, methanesulfonate salt crystalline form is characterized by an XRPD pattern substantially as shown in FIG. 8 and a TGA curve substantially as shown in FIG. 9.

In some embodiments, the compound of Formula I, methanesulfonate salt crystalline form is a solvate.

In some embodiments, the compound of Formula I, methanesulfonate salt crystalline form is not a solvate.

### Compound of Formula I, Ethanesulfonate Slat and Crystalline Forms Thereof

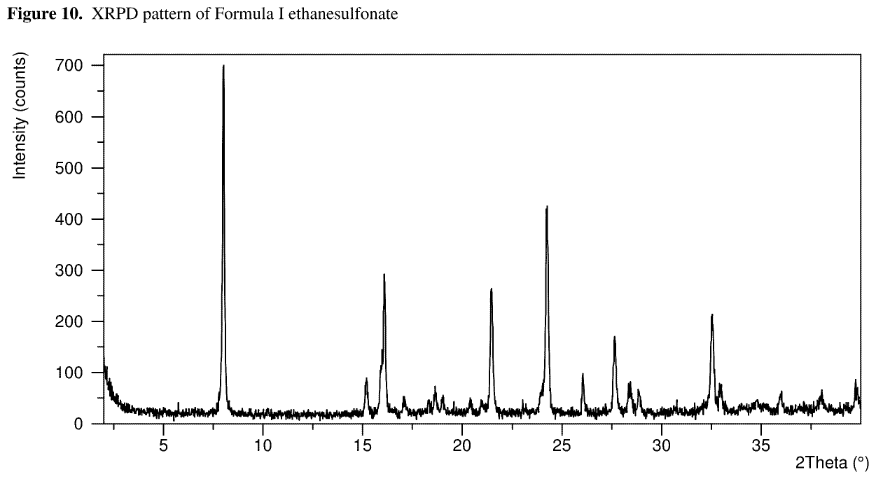

In some embodiments, the present disclosure provides a ethanesulfonate salt of the compound of Formula I. In some embodiments, the compound of Formula I, ethanesulfonate salt is crystalline. In some embodiments, the compound of Formula I, ethanesulfonate salt is a crystalline form having an XRPD profile substantially as shown in FIG. 10. In some embodiments, compound of Formula I, ethanesulfonate salt is a crystalline form having an XRPD pattern displaying at least two, at least three, at least four, at least five, or at least six of the 2θ-reflections as the XRPD pattern substantially as shown in FIG. 10.

In some embodiments, the compound of Formula I, ethanesulfonate salt crystalline form is characterized by an XRPD pattern comprising 2θ-reflections (±0.2 degrees 2θ) at about 8.0°, 16.1°, and 32.5°. In some embodiments, compound of Formula I, ethanesulfonate salt crystalline form has an XRPD pattern comprising 2θ-reflections (±0.2 degrees 2θ) at 8.0°, 16.1°, and 32.5°, and one, two or three of the 2θ-reflections (±0.2 degrees 2θ) at 15.2°, 21.5°, and 27.6°. In some embodiments, the compound of Formula I, ethanesulfonate salt crystalline form has an XRPD pattern comprising 2θ-reflections (±0.2 degrees 2θ) at 8.0°, 16.1°, and 32.5°, and one or two of the 2θ-reflections (±0.2 degrees 2θ) at 15.2°, 21.5°, and 27.6°. In some embodiments, the compound of Formula I, ethanesulfonate salt crystalline form has an XRPD pattern comprising 2θ-reflections (±0.2 degrees 2θ) at 8.0°, 16.1°, and 32.5°, and one of the 2θ-reflections (±0.2 degrees 2θ) at 15.2°, 21.5°, and 27.6°. In some embodiments, the compound of Formula I, ethanesulfonate salt crystalline form has an XRPD pattern comprising 2θ-reflections (±0.2 degrees 2θ) at 8.0°, 16.1°, and 32.5°, and two of the 2θ-reflections (±0.2 degrees 2θ) at 15.2°, 21.5°, and 27.6°. In some embodiments, the compound of Formula I, ethanesulfonate salt crystalline form has an XRPD pattern comprising 2θ-reflections (±0.2 degrees 2θ) at 8.0°, 15.2°, 16.1°, 21.5°, 27.6°, and 32.5°. In some embodiments, the compound of Formula I, ethanesulfonate salt crystalline form has an XRPD pattern comprising any three 2θ-reflections (±0.2 degrees 2θ) selected from the group consisting of 8.0°, 15.2°, 16.1°, 21.5°, 27.6°, and 32.5°

In some embodiments, the compound of Formula I, ethanesulfonate salt crystalline form has an XRPD pattern comprising 2θ-reflections (±0.2 degrees 2θ) at 8.0°, 15.2°, 16.1°, 21.5°, 27.6°, and 32.5°, and one, two or three of the 2θ-reflections (±0.2 degrees 2θ) at 18.6°, 26.1°, and 33.0°. In some embodiments, the compound of Formula I, ethanesulfonate salt crystalline form has an XRPD pattern comprising 2θ-reflections (±0.2 degrees 2θ) at 8.0°, 15.2°, 16.1°, 21.5°, 27.6°, and 32.5° and one or two of the 2θ-reflections (±0.2 degrees 2θ) at 18.6°, 26.1°, and 33.0°. In some embodiments, the compound of Formula I, ethanesulfonate salt crystalline form has an XRPD pattern comprising 2θ-reflections (±0.2 degrees 2θ) at 8.0°, 15.2°, 16.1°, 21.5°, 27.6°, and 32.5°, and one of the 2θ-reflections (±0.2 degrees 2θ) at 18.6°, 26.1°, and 33.0°. In some embodiments, the compound of Formula I, ethanesulfonate salt crystalline form has an XRPD pattern comprising 2θ-reflections (±0.2 degrees 2θ) at 8.0°, 15.2°, 16.1°, 21.5°, 27.6°, and 32.5°, and two of the 2θ-reflections (±0.2 degrees 2θ) at 18.6°, 26.1°, and 33.0°. In some embodiments, the compound of Formula I, ethanesulfonate salt crystalline form has an XRPD pattern comprising 2θ-reflections (±0.2 degrees 2θ) at 8.0°, 15.2°, 16.1°, 18.6°, 21.5°, 26.1°, 27.6°, 32.5°, and 33.0°. In some embodiments, the compound of Formula I, ethanesulfonate salt crystalline form has an XRPD pattern comprising any three 2θ-reflections (±0.2 degrees 2θ) selected from the group consisting of 8.0°, 15.2°, 16.1°, 18.6°, 21.5°, 26.1°, 27.6°, 32.5°, and 33.0°.

In some embodiments, the compound of Formula I, ethanesulfonate salt crystalline form has a XRPD pattern comprising peaks at:

| Pos. [°2Th.] | Rel. Int. [%] |
|---|---|
| 8.0 | 100 |
| 15.2 | 7 |
| 16.1 | 41 |
| 17.0 | 3 |
| 18.6 | 6 |
| 20.4 | 2 |
| 21.5 | 29 |
| 24.2 | 60 |
| 26.1 | 7 |
| 27.6 | 15 |
| 28.4 | 4 |
| 28.9 | 3 |
| 32.5 | 31 |
| 33.0 | 4 |
| 34.7 | 2 |
| 35.9 | 4 |
| 38.1 | 3 |

### Methods of Preparation

The present disclosure also provides methods of making the solid forms disclosed herein.

In some embodiments, the present disclosure provides a method of making a salt of a compound of Formula I:

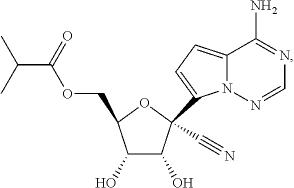 [SMILES: CC(C)C(=O)OC[C@H]1O[C@@](C#N)(c2ccc3c(N)ncnn23)[C@H](O)[C@@H]1O]

wherein the method comprises treating the compound of Formula I with an acid, wherein the salt is a maleate, oxalate, methanesulfonate, or ethanesulfonate; and wherein the acid is maleic acid, oxalic acid, methanesulfonic acid, or ethanesulfonic acid.

In some embodiments, the disclosure provides a method of making a maleate salt of a compound of Formula I, the method comprising adding maleic acid to the compound of Formula I.

In some embodiments, the disclosure provides a method of making a crystalline form of the maleate salt of a compound of Formula I, the method comprising adding maleic acid to the compound of Formula I. In some embodiments, the maleic acid is added to a crystalline Form III of the compound of Formula I, wherein the crystalline Form III is characterized by an XRPD pattern comprising degree 2θ-reflections (+/−0.2 degrees 2θ) at 9.8°, 16.0°, and 25.4°. In some embodiments, one molar equivalent of maleic acid is added to a crystalline Form III of the compound of Formula I.

In some embodiments, the disclosure provides a method of making an oxalate salt of a compound of Formula I, the method comprising adding oxalic acid to the compound of Formula I.

In some embodiments, the disclosure provides a method of making a crystalline form of an oxalate salt of a compound of Formula I, the method comprising adding oxalic acid to the compound of Formula 1. In some embodiments, oxalic acid is added to a crystalline Form III of the compound of Formula I, wherein the crystalline Form III is characterized by an XRPD pattern comprising degree 2θ-reflections (+/−0.2 degrees 2θ) at 9.8°, 16.0°, and 25.4°. In some embodiments, about two molar equivalents of oxalic acid is added to a crystalline Form III of the compound of Formula I.

In some embodiments, the disclosure provides a method of making a methanesulfonate salt of a compound of Formula I, the method comprising adding methanesulfonic acid to the compound of Formula I.

In some embodiments, the disclosure provides a method of making a crystalline form of an methanesulfonate salt of a compound of Formula I, the method comprising adding methanesulfonic acid to the compound of Formula I. In some embodiments, methanesulfonic acid is added to a crystalline Form III of the compound of Formula 1, wherein the crystalline Form III is characterized by an XRPD pattern comprising degree 2θ-reflections (+/−0.2 degrees 2θ) at 9.8°, 16.0°, and 25.4°. In some embodiments, about one molar equivalents of methanesulfonic acid is added to a crystalline Form III of the compound of Formula I. In some embodiments, about 1.1 molar equivalents of methanesulfonic acid is added to a crystalline Form III of the compound of Formula I.

In some embodiments, the disclosure provides a method of making method of making an ethanesulfonate salt of a compound of Formula I, the method comprising adding ethanesulfonic acid to the compound of Formula I.

In some embodiments, the disclosure provides a method of making method of making a crystalline form of an ethanesulfonate salt of a compound of Formula I, the method comprising adding ethanesulfonic acid to the compound of Formula I. In some embodiments, ethanesulfonic acid is added to a crystalline Form III of the compound of Formula I, wherein the crystalline Form III is characterized by an XRPD pattern comprising degree 2θ-reflections (+/−0.2 degrees 2θ) at 9.8°, 16.0°, and 25.4°. In some embodiments, about one molar equivalents of ethanesulfonic acid is added to a crystalline Form III of the compound of Formula I.

### Pharmaceutical Compositions

The solid forms disclosed herein may be formulated with conventional carriers and excipients. For example, tablets will contain excipients, glidants, fillers, binders and the like. Aqueous formulations are prepared in sterile form, and when intended for delivery by other than oral administration generally will be isotonic. All formulations may optionally comprise excipients such as those set forth in the “Handbook of Pharmaceutical Excipients” (1986). Pharmaceutically acceptable excipients include ascorbic acid and other antioxidants, chelating agents such as EDTA, carbohydrates such as dextran, hydroxyalkylcellulose, hydroxyalkylmethylcellulose, stearic acid and the like. In some embodiments, the formulations comprise one or more pharmaceutically acceptable excipients. The pH of the formulations ranges from about 3 to about 11, but is ordinarily about 7 to 10. In some embodiments, the pH of the formulations ranges from about 2 to about 5, but is ordinarily about 3 to 4.

While it is possible for the solid forms of the disclosure (“the active ingredients”) to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any appropriate method known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In some embodiments, the pharmaceutical formulation is for subcutaneous, intramuscular, intravenous, oral, or inhalation administration.

In some embodiments, the solid forms described herein have optimized/improved pharmacokinetic properties and are amenable to oral administration. For example, the solid forms disclosed herein have improved bioavailability and can therefore be administered by oral administration.

In some embodiments, the formulations of the present invention are suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste,

In some embodiments, the tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation of the invention include Tween® 80.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a solid form disclosed herein together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight: weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the solid forms disclosed herein are administered by inhalation. In some embodiments, formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the solid forms used herein are formulated and dosed as dry powder. In some embodiments, the solid forms used herein are formulated and dosed as a nebulized formulation. In some embodiments, the solid forms used herein are formulated for delivery by a face mask. In some embodiments, the solid forms used herein are formulated for delivery by a face tent.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Solid forms of the invention are used to provide controlled release pharmaceutical formulations containing as active ingredient one or more solid forms of the invention (“controlled release formulations”) in which the release of the active ingredient are controlled and regulated to allow less frequency dosing or to improve the pharmacokinetic or toxicity profile of a given active ingredient.

### Kits

Also provided herein are kits that includes a solid form disclosed herein. In some embodiments the kits described herein may comprise a label and/or instructions for use of the solid form in the treatment of a disease or condition in a subject (e.g., human) in need thereof. In some embodiments, the disease or condition is viral infection.

In some embodiments, the kit may also comprise one or more additional therapeutic agents and/or instructions for use of additional therapeutic agents in combination with the solid form disclosed herein in the treatment of the disease or condition in a subject (e.g., human) in need thereof.

In some embodiments, the kits provided herein comprises individual dose units of a solid form as described herein. Examples of individual dosage units may include pills, tablets, capsules, prefilled syringes or syringe cartridges, IV bags, inhalers, nebulizers etc., each comprising a therapeutically effective amount of the solid form in question. In some embodiments, the kit may contain a single dosage unit and in others, multiple dosage units are present, such as the number of dosage units required for a specified regimen or period.

Also provided are articles of manufacture that include a solid form disclosed herein and a container. In some embodiments, the container of the article of manufacture is a vial, jar, ampoule, preloaded syringe, blister package, tin, can, bottle, box, an intravenous bag, an inhaler, or a nebulizer.

### Administration

One or more solid forms of the disclosure are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, solid form disclosed herein are administered by inhalation or intravenously. In some embodiments, the solid form disclosed herein are administered orally. It will be appreciated that the preferred route may vary with for example the condition of the recipient.

In the methods of the present invention for the treatment of a viral infection, the solid form disclosed herein can be administered at any time to a human who may come into contact with the virus or is already suffering from the viral infection. In some embodiments, the solid form disclosed herein can be administered prophylactically to humans coming into contact with humans suffering from the viral infection or at risk of coming into contact with humans suffering from the viral infection, e.g., healthcare providers. In some embodiments. administration of the solid form disclosed herein can be to humans testing positive for the viral infection but not yet showing symptoms of the viral infection. In some embodiments, administration of the solid form disclosed herein can be to humans upon commencement of symptoms of the viral infection.

In some embodiments, the methods disclosed herein comprise event driven administration of the solid form disclosed herein to the subject.

As used herein, the terms “event driven” or “event driven administration” refer to administration of the solid form described herein, (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, a solid form disclosed herein is administered before exposure of the subject to the virus.

In some embodiments, a solid form disclosed herein is administered before and after exposure of the subject to the virus.

In some embodiments, a solid form disclosed herein is administered after exposure of the subject to the virus.

An example of event driven dosing regimen includes administration a solid form disclosed herein within 24 to 2 hours prior to the virus, followed by administration of a solid form disclosed herein every 24 hours during the period of exposure, followed by a further administration of a solid form disclosed herein after the last exposure, and one last administration of a solid form disclosed herein 24 hours later.

A further example of an event driven dosing regimen includes administration of a solid form disclosed herein within 24 hours before the viral exposure, then daily administration during the period of exposure, followed by a last administration approximately 24 hours later after the last exposure (which may be an increased dose, such as a double dose).

The specific dose level of a solid form disclosed herein for any particular subject will depend upon a variety of factors including the activity of the specific solid form employed, the age, body weight, general health, sex, diet, time of administration, route of administration, and rate of excretion, drug combination and the severity of the particular disease in the subject undergoing therapy. For example, a dosage may be expressed as a number of milligrams of a solid form disclosed herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a solid form disclosed herein administered per dose or per day. Daily dosage of a solid form disclosed herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a solid form disclosed herein may be adjusted over the course of the treatment, based on the judgment of the administering physician.

The solid forms disclosed herein may be administered to an individual (e.g., a human) in a therapeutically effective amount. In some embodiments, the solid forms are administered once daily.

The solid forms disclosed herein can be administered by any useful route and means, such as by oral or parenteral (e.g., intravenous) administration. Therapeutically effective amounts of the solid forms may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the solid forms provided herein include from about 0.3 mg to about 30 mg per day, or from about 30 mg to about 300 mg per day, or from about 0.3 mg to about 30 mg per day, or from about 30 mg to about 300 mg per day.

A solid forms of the present disclosure may be combined with one or more additional therapeutic agents in any dosage amount of the solid form of the present disclosure (e.g., from 1 mg to 1000 mg of solid form). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the solid forms are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the solid forms of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a solid form provided herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day. In some embodiments, the total daily dosage for a human subject is about 500-1000 mg/day administered once or twice daily.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

In some embodiments, the total daily dosage for a human subject may be about 100 mg/day administered in two dose daily. In some embodiments, the total daily dosage for a human subject may be about 150 mg/day administered in two dose daily. In some embodiments, the total daily dosage for a human subject may be about 200 mg/day administered in two dose daily. In some embodiments, the total daily dosage for a human subject may be about 250 mg/day administered in two dose daily. In some embodiments, the total daily dosage for a human subject may be about 300 mg/day administered in two dose daily. In some embodiments, the total daily dosage for a human subject may be about 350 mg/day administered in two dose daily. In some embodiments, the total daily dosage for a human subject may be about 400 mg/day administered in two dose daily. In some embodiments, the total daily dosage for a human subject may be about 450 mg/day administered in two dose daily. In some embodiments, the total daily dosage for a human subject may be about 500 mg/day administered in two dose daily. In some embodiments, the total daily dosage for a human subject may be about 550 mg/day administered in two dose daily. In some embodiments, the total daily dosage for a human subject may be about 600 mg/day administered in two dose daily. In some embodiments, the total daily dosage for a human subject may be about 650 mg/day administered in two dose daily. In some embodiments, the total daily dosage for a human subject may be about 700 mg/day administered in two dose daily. In some embodiments, the total daily dosage for a human subject may be about 750 mg/day administered in two dose daily. In some embodiments, the total daily dosage for a human subject may be about 800 mg/day administered in two dose daily. In some embodiments, the total daily dosage for a human subject may be about 850 mg/day administered in two dose daily. In some embodiments, the total daily dosage for a human subject may be about 900 mg/day administered in two dose daily. In some embodiments, the total daily dosage for a human subject may be about 950 mg/day administered in two dose daily. In some embodiments, the total daily dosage for a human subject may be about 1000 mg/day administered two dose daily. In some embodiments, the total daily dosage for a human subject may be about 1500 mg/day administered two dose daily. In some embodiments, the total daily dosage for a human subject may be about 2000 mg/day administered two dose daily. In some embodiments, the total daily dosage for a human subject may be about 2500 mg/day administered two dose daily. In some embodiments, the total daily dosage for a human subject may be about 3000 mg/day administered two dose daily. In some embodiments, the total daily dosage for a human subject may be about 4000 mg/day administered two dose daily.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a solid form disclosed herein is administered once daily in a method disclosed herein. In some embodiments, a solid form disclosed herein is administered twice daily in a method disclosed herein. In some embodiments, a solid form disclosed herein is administered three times daily in a method disclosed herein.

In some embodiments, a solid form disclosed herein is administered once daily in the total daily dose of 100-4000 mg/day. In some embodiments, a solid form disclosed herein is administered twice daily in the total daily dose of 100-4000 mg/day. In some embodiments, a solid form disclosed herein is administered three times daily in the total daily dose of 100-4000 mg/day. In some embodiments, a solid form disclosed herein is administered once daily in the total daily dose of 300-900 mg/day.

The frequency of dosage of the solid form of the present disclosure will be determined by the needs of the individual patient and can be, for example, once per day or twice, or more times, per day. Administration of the solid form continues for as long as necessary to treat the viral infection. For example, a solid form can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of a solid form of the present disclosure followed by a period of several or more days during which a patient does not receive a daily dose of the solid form. For example, a patient can receive a dose of the solid form every other day, or three times per week. Again by way of example, a patient can receive a dose of the solid form each day for a period of from 1 to 14 days, followed by a period of 7 to 21 days during which the patient does not receive a dose of the solid form, followed by a subsequent period (e.g., from 1 to 14 days) during which the patient again receives a daily dose of the solid form. Alternating periods of administration of the solid form, followed by non-administration of the solid form, can be repeated as clinically required to treat the patient.

The solid forms of the present disclosure or the pharmaceutical compositions thereof may be administered once, twice, three, or four times daily, using any suitable mode described above. Also, administration or treatment with the solid forms may be continued for a number of days; for example, commonly treatment would continue for at least 3 days, at least 5 days, at least 7 days, 14 days, or 28 days, for one cycle of treatment.

### Methods of Use

The present disclosure also provides a method of treating or preventing a viral infection in a subject (e.g., human) in need thereof, the method comprising administering to the subject a solid form described herein.

In some embodiments, the present disclosure provides a method of treating a viral infection in a subject (e.g., human) in need thereof, the method comprising administering to a subject in need thereof a solid form described herein.

In some embodiments, the solid form described herein is administered to the human via oral, intramuscular, intravenous, subcutaneous, or inhalation administration.

In some embodiments, the present disclosure provides for methods of treating or preventing a viral infection in a subject (e.g., human) in need thereof, the method comprising administering to the subject a solid form disclosed herein and at least one additional active therapeutic or prophylactic agent.

In some embodiments, the present disclosure provides for methods of treating a viral infection in a subject (e.g., human) in need thereof, the method comprising administering to the subject a solid form disclosed herein, and at least one additional active therapeutic or prophylactic agent.

In one embodiment, the present disclosure provides for methods of inhibiting a viral polymerase in a cell, the methods comprising contacting the cell infected by a virus with a solid form disclosed herein, whereby the viral polymerase is inhibited.

In one embodiment, the present disclosure provides for methods of inhibiting a viral polymerase in a cell, the methods comprising contacting the cell infected by a virus with a solid form disclosed herein, and at least one additional active therapeutic agent, whereby the viral polymerase is inhibited.

Also provided here are the uses of the solid forms disclosed herein for use in treating or preventing a viral infection in a subject in need thereof. For example, provided herein are uses of the solid forms disclosed herein for use in treating a viral infection in a subject in need thereof.

In some embodiments, the viral infection is a paramyxoviridae virus infection. As such, in some embodiments, the present disclosure provides methods for treating a paramyxoviridae infection in a subject (e.g., a human) in need thereof, the method comprising administering to the subject a solid form disclosed herein. Paramyxoviridae viruses include, but are not limited to Nipah virus, Hendra virus, measles, mumps, and parainfluenze virus.

In some embodiments, the viral infection is a human parainfluenza virus, Nipah virus, Hendra virus, measles, or mumps infection.

In some embodiments, the viral infection is a pneumoviridae virus infection. As such, in some embodiments, the present disclosure provides a method of treating a pneumoviridae virus infection in a human in need thereof, the method comprising administering to the human a solid form provided herein. Pneumoviridae viruses include, but are not limited to, respiratory snycytial virus and human metapneumovirus. In some embodiments, the pneumoviridae virus infection is a respiratory syncytial virus infection. In some embodiments, the pneumoviridae virus infection is human metapneumovirus infection.

In some embodiments, the present disclosure provides a solid form disclosed herein, for use in the treatment of a pneumoviridae virus infection in a human in need thereof. In some embodiments, the pneumoviridae virus infection is a respiratory syncytial virus infection. In some embodiments, the pneumoviridae virus infection is human metapneumovirus infection.

In some embodiments, the present disclosure provides methods for treating a RSV infection in a human in need thereof, the method comprising administering to the human a solid form provided herein. In some embodiments, the human is suffering from a chronic respiratory syncytial viral infection. In some embodiments, the human is acutely infected with RSV.

In some embodiments, a method of inhibiting RSV replication is provided, wherein the method comprises administering to a human in need thereof, a solid form disclosed herein, wherein the administration is by inhalation.

In some embodiments, the present disclosure provides a method for reducing the viral load associated with RSV infection, wherein the method comprises administering to a human infected with RSV a solid form disclosed herein.

In some embodiments, the viral infection is a picornaviridae virus infection. As such, in some embodiments, the present disclosure provides a method of treating a picornaviridae virus infection in a human in need thereof, the method comprising administering to the human a solid form of the present disclosure. Picornaviridae viruses are enteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection (HRV). In some embodiments, the Picornaviridae virus infection is HRV-A, HRV-B, or HRV-C infection.

In some embodiments, the viral infection is selected from the group consisting of Coxsackie A virus infection, Coxsackie A virus infection, enterovirus D68 infection, enterovirus B69 infection, enterovirus D70 infection, enterovirus A71 infection, and poliovirus infection.

In some embodiments, the present disclosure provides a solid form, for use in the treatment of a picornaviridae virus infection in a human in need thereof. In some embodiments, the picornaviridae virus infection is human rhinovirus infection.

In some embodiments, the viral infection is a flaviviridae virus infection. As such, in some embodiments, the present disclosure provides a method of treating a flaviviridae virus infection in a human in need thereof, the method comprising administering to the human a solid form described herein. Representative flaviviridae viruses include, but are not limited to, dengue, Yellow fever, West Nile, Zika, Japanese encephalitis virus, and Hepatitis C (HCV). In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a Japanese ensephalitis virus infection. In some embodiments, the flaviviridae virus infection is a hepatitis C virus infection.

In some embodiments, the flaviviridae virus infection is a dengue virus infection, yellow fever virus infection, West Nile virus infection, tick borne encephalitis, Kunjin Japanese encephalitis, St. Louis encephalitis, Murray valley encephalitis, Omsk hemorrhagic fever, bovine viral diarrhea, zika virus infection, or a HCV infection.

In some embodiments, the present disclosure provides use of a solid form disclosed herein for treatment of a flaviviridae virus infection in a human in need thereof. In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a hepatitis C virus infection.

In some embodiments, the viral infection is a filoviridae virus infection. As such, in some embodiments, provided herein is a method of treating a filoviridae virus infection in a human in need thereof, the method comprising administering to the human a solid form disclosed herein. Representative filoviridae viruses include, but are not limited to, ebola (variants Zaire, Bundibugio, Sudan, Tai forest, or Reston) and marburg. In some embodiments, the filoviridae virus infection is an ebola virus infection. In some embodiments, the filoviridae virus infection is a marburg virus infection.

In some embodiments, the present disclosure provides a solid form for use in the treatment of a filoviridae virus infection in a human in need thereof. In some embodiments, the filoviridae virus infection is an ebola virus infection. In some embodiments, the filoviridae virus infection is a marburg virus infection.

In some embodiments, the viral infection is a coronavirus infection. As such, in some embodiments, provided herein is a method of treating a coronavirus infection in a human in need thereof, wherein the method comprises administering to the human a solid form provided herein. In some embodiments, the coronavirus infection is a Severe Acute Respiratory Syndrome (SARS-COV) infection, Middle Eastern Respiratory Syndrome (MERS) infection, SARS-COV-2 infection, other human coronavirus (229E, NL63, OC43, HKU1, or WIV1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-COV-2 infection. In some embodiments, the viral infection is a zoonotic coronavirus infection, In some embodiments, the viral infection is caused by a virus having at least 70% sequence homology to a viral polymerase selected from the group consisting of SARS-COV polymerase, MERS-COV polymerase and SARS-COV-2. In some embodiments, the viral infection is caused by a virus having at least 80% sequence homology to a viral polymerase selected from the group consisting of SARS-COV polymerase, MERS-COV polymerase and SARS-COV-2. In some embodiments, the viral infection is caused by a virus having at least 90% sequence homology to a viral polymerase selected from the group consisting of SARS-COV polymerase, MERS-COV polymerase and SARS-COV-2. In some embodiments, the viral infection is caused by a virus having at least 95% sequence homology to a viral polymerase selected from the group consisting of SARS-COV polymerase, MERS-COV polymerase and SARS-COV-2.

In some embodiments, the viral infection is caused by a variant of SARS-COV-2, for example by the B.1.1.7 variant (the UK variant), B. 1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-COV-2. In some embodiments, the viral infection is caused by the B. 1.351 variant of SARS-COV-2. In some embodiments, the viral infection is caused by the P.1 variant of SARS-COV-2.

In some embodiments, the present disclosure provides a solid form for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-COV-2 infection, other human coronavirus (229E, NL63, OC43, HKU1, or WIV1) infections, and zoonotic coronavirus (PEDV or HKU CoV isolates such as HKU3, HKUS, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-COV-2 infection (COVID19).

In some embodiments, the viral infection is an arenaviridae virus infection. As such, in some embodiments, the disclosure provides a method of treating an arenaviridae virus infection in a human in need thereof, the method comprising administering to the human a solid form disclosed herein. In some embodiments, the arenaviridae virus infection is a Lassa infection or a Junin infection.

In some embodiments, the present disclosure provides a solid form for use in the treatment of an arenaviridae virus infection in a human in need thereof. In some embodiments, the arenaviridae virus infection is a Lassa infection or a Junin infection.

In some embodiments, the viral infection is an orthomyxovirus infection, for example, an influenza virus infection. In some embodiments, the viral infection is an influenza virus A, influenza virus B, or influenza virus C infection.

In some embodiments, the viral infection is a buynavirale infection. As such, in some embodiments, the present disclosure provides methods for treating a buynavirale infection in a subject (e.g., a human) in need thereof, the method comprising administering to the subject a solid form disclosed herein. Buynavirale infections include, but are not limited to phenuiviridae (e.g. Rift Valley fever virus), arenaviridae (e.g. Lassa and Junin as described above), nairoviridae (e.g. Crimean-Congo hemorrhagic fever (CCHF) virus), and hantaviridae (e.g. hantaviruses) infections.

In some embodiments, the viral infection is a poxvirus infection. As such, in some embodiments, the present disclosure provides methods for treating a poxvirus infection in a subject (e.g., a human) in need thereof, the method comprising administering to the subject a compound disclosed herein. In some embodiments, the present disclosure provides methods for treating a poxvirus infection in a subject (e.g., a human) in need thereof, the method comprising administering to the subject a solid form disclosed herein.

In some embodiments, the poxvirus infection is an orthopox virus infection. In some embodiments, the poxvirus infection is a camelpox virus infection, cowpox virus infection, ectromelia virus infection, horsepox virus infection, monkeypox virus infection, raccoonpox virus infection, skunkpox virus infection, taterapox virus infection, uasin gishu virus infection, vaccinia virus infection, variola virus infection, or volepox virus infection.

In some embodiments, the poxvirus infection is a vaccinia virus infection.

In some embodiments, the poxvirus infection is a monkeypox virus infection. The methods described herein can be used to treat or prevent an infection caused by any strain of monkeypox virus. In some embodiments, the poxvirus infection is caused by a West African strain of monkeypox virus. In some embodiments, the poxvirus infection is caused by a Congo Basin strain of monkeypox virus.

In some embodiments, the poxvirus infection is a parapoxvirus infection. In some embodiments, the poxvirus infection is bovine papular stomatitis virus infection, orf virus infection, pseudocowpox virus infection, parapoxvirus of red deer infection, or squirrel parapoxvirus infection. In some embodiments, the poxvirus infection is camel contagious ecthyma (Ausdyk) virus infection, chamois contagious ecthyma virus infection, parapoxvirus of reindeer virus infection, or sealpox virus infection.

In some embodiments, the poxvirus infection is a molluscipoxvirus infection. In some embodiments, the poxvirus infection is a molluscum contagiosum infection.

In some embodiments, the poxvirus infection is a yatapoxvirus infection. In some embodiments, the poxvirus infection is a Tanapox, Yaba-like disease virus infection or yaba monkey tumor virus infection.

In some embodiments, the poxvirus infection is a capripoxvirus infection. In some embodiments, the poxvirus infection is a sheeppox virus infection, goatpox virus infection, or lumpy skin disease virus infection.

In some embodiments, the poxvirus infection is a suipoxvirus infection. In some embodiments, the poxvirus infection is a swinepox virus infection.

In some embodiments, the poxvirus infection is a leporipoxvirus infection. In some embodiments, the poxvirus infection is a myxoma virus infection, shope fibroma virus (rabbit fibroma) infection, squirrel fibroma virus infection, or hare fibroma virus infection.

In some embodiments, the poxvirus infection is an avipoxvirus infection. In some embodiments, the poxvirus infection is canarypox virus infection, fowlpox virus infection, juncopox virus infection, mynahpox virus infection, pigeonpox virus infection, psittacinepox virus infection, quailpox virus infection, sparrowpox virus infection, starlingpox virus infection, or turkeypox virus infection. In some embodiments, the poxvirus infection is crowpox virus infection, peacockpox virus infection, or penguinpox virus infection.

As described more fully herein, the solid forms described herein can be administered with one or more additional therapeutic agent(s) to an individual (e.g., a human) infected with a viral infection. The additional therapeutic agent(s) can be administered to the infected individual at the same time as the compound of the present disclosure or before or after administration of the compound of the present disclosure.

### Combination Therapy

The solid forms described herein can also be used in combination with one or more additional therapeutic agents. As such, also provided herein are methods of treatment of a viral infection in a subject in need thereof, wherein the methods comprise administering to a subject in need thereof a solid form of the disclosure and a therapeutically effective amount of one or more additional therapeutic agents.

In some embodiments, the additional therapeutic agent is an antiviral agent. Any suitable antiviral agent can be used in the methods described herein. In some embodiments, the antiviral agent is selected from 5-substituted 2′-deoxyuridine analogues, nucleoside analogues, pyrophosphate analogues, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A/NS5B inhibitors, influenza virus inhibitors, interferons, immunostimulators, oligonucleotides, antimitotic inhibitors, and combinations thereof.

In some embodiments, the additional therapeutic agent is a 5-substituted 2′-deoxyuridine analogue. For example, in some embodiments, the additional therapeutic agent is selected from idoxuridine, trifluridine, brivudine [BVDU], and combinations thereof.

In some embodiments, the additional therapeutic agent is a nucleoside analogue. For example, in some embodiments, the additional therapeutic agent is selected from vidarabine, entecavir (ETV), telbivudine, lamivudine, adefovir dipivoxil, tenofovir disoproxil fumarate (TDF) and combinations thereof. In some embodiments, the additional therapeutic agent is favipiravir, ribavirin, galidesivir, β-D-N4-hydroxycytidine or a combination thereof.

In some embodiments, the additional therapeutic agent is a pyrophosphate analogue. For example, in some embodiments, the additional therapeutic agent is foscarnet or phosphonoacetic acid. In some embodiments, the additional therapeutic agent is foscarnet.

In some embodiments, the additional therapeutic agent is nucleoside reverse transcriptase inhibitor. In some embodiments, the antiviral agent is zidovudine, didanosine, zalcitabine, stavudine, lamivudine, abacavir, emtricitabine, and combinations thereof.

In some embodiments, the additional therapeutic agent is a non-nucleoside reverse transcriptase inhibitor. In some embodiments, the antiviral agent is selected from nevirapine, delavirdine, efavirenz, etravirine, rilpivirine, and combinations thereof.

In some embodiments, the additional therapeutic agent is a protease inhibitor. In some embodiments, the protease inhibitor is a HIV protease inhibitor. For example, in some embodiments, the antiviral agent is selected from saquinavir, ritonavir, indinavir, nelfinavir, amprenavir, lopinavir, atazanavir, fosamprenavir, darunavir, tipranavir, cobicistat, and combinations thereof. In some embodiments, the antiviral agent is selected from saquinavir, ritonavir, indinavir, nelfinavir, amprenavir, lopinavir, atazanavir, fosamprenavir, darunavir, tipranavir, and combinations thereof. In some embodiments, the protease inhibitor is a HCV NS3/4A protease inhibitor. For example, in some embodiments, the additional therapeutic agent is selected from voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir and combinations thereof. In some embodiments, the additional therapeutic agent is selected from voxilaprevir, asunaprevir, boceprevir, paritaprevir, simeprevir, telaprevir, vaniprevir, grazoprevir, and combinations thereof.

In some embodiments, the additional therapeutic agent is an integrase inhibitor. For example, in some embodiments, the additional therapeutic agent is selected from raltegravir, dolutegravir, elvitegravir, abacavir, lamivudine, and combinations thereof. In some embodiments, the additional therapeutic agent is selected from bictegravir, raltegravir, dolutegravir, cabotegravir, elvitegravir, and combinations thereof. In some embodiments, the additional therapeutic agent is selected from bictegravir, dolutegravir, and cabotegravir, and combinations thereof. In some embodiments, the additional therapeutic agent is bictegravir.

In some embodiments, the additional therapeutic agent is an entry inhibitor. For example, in some embodiments, the additional therapeutic agent is selected from docosanol, enfuvirtide, maraviroc, ibalizumab, fostemsavir, leronlimab, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), and combinations thereof.

In some embodiments, the additional therapeutic agent is an acyclic guanosine analogue. For example, in some embodiments, the additional therapeutic agent is selected from acyclovir, ganciclovir, valacyclovir (also known as valaciclovir), valganciclovir, penciclovir, famciclovir, and combinations thereof.

In some embodiments, the additional therapeutic agent is an acyclic nucleoside phosphonate analogue. For example, in some embodiments, the additional therapeutic agent is selected from a group consisting of cidofovir, adefovir, adefovir dipivoxil, tenofovir, TDF, emtricitabine, efavirenz, rilpivirine, elvitegravir, and combinations thereof. In some embodiment, the additional therapeutic agent is selected from cidofovir, adefovir, adefovir dipivoxil, tenofovir, TDF, and combinations thereof. In some embodiment, the additional therapeutic agent is selected from cidofovir, adefovir dipivoxil, TDF, and combinations thereof.

In some embodiments, the additional therapeutic agent is a HCV NSSA/NS5B inhibitor. In some embodiments, the additional therapeutic agent is a NS3/4A protease inhibitor. In some embodiments, the additional therapeutic agent is a NSSA protein inhibitor. In some embodiments, the additional therapeutic agent is a NS5B polymerase inhibitor of the nucleoside/nucleotide type. In some embodiments, the additional therapeutic agent is a NSSB polymerase inhibitor of the nonnucleoside type. In some embodiments, the additional therapeutic agent is selected from daclatasvir, ledipasvir, velpatasvir, ombitasvir, elbasvir, sofosbuvir, dasabuvir, ribavirin, asunaprevir, simeprevir, paritaprevir, ritonavir, elbasvir, grazoprevir, AT-527, and combinations thereof. In some embodiments, the additional therapeutic agent is selected from daclatasvir, ledipasvir, velpatasvir, ombitasvir, elbasvir, sofosbuvir, dasabuvir, and combinations thereof.

In some embodiments, the additional therapeutic agent is an influenza virus inhibitor. In some embodiments, the additional therapeutic agent is a matrix 2 inhibitor. For example, in some embodiments, the additional therapeutic agent is selected from amantadine, rimantadine, and combinations thereof. In some embodiments, the additional therapeutic agent is a neuraminidase inhibitor. For example, in some embodiments, the additional therapeutic agent is selected from zanamivir, oseltamivir, peramivir, laninamivir octanoate, and combinations thereof. In some embodiments, the additional therapeutic agent is a polymerase inhibitor. For example, in some embodiments, the additional therapeutic agent is selected from ribavirin, favipiravir, and combinations thereof. In some embodiments, the additional therapeutic agent is selected from amantadine, rimantadine, arbidol (umifenovir), baloxavir marboxil, oseltamivir, peramivir, ingavirin, laninamivir octanoate, zanamivir, favipiravir, ribavirin, and combinations thereof. In some embodiments, the additional therapeutic agent is selected from amantadine, rimantadine, zanamivir, oseltamivir, peramivir, laninamivir octanoate, ribavirin, favipiravir, and combinations thereof.

In some embodiments, the additional therapeutic agent is an interferon. In some embodiments, the additional therapeutic agent is selected from interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b. e embodiments, the additional therapeutic agent is selected from interferon alfacon 1, interferon alfa 1b, interferon alfa 2a, interferon alfa 2b, pegylated interferon alfa 2a (PegIFNα-2a), and PegIFNα-2b. In some embodiments, the additional therapeutic agent is selected from interferon alfacon 1, pegylated interferon alfa 2a (PegIFNα-2a), PegIFNα-2b, and ribavirin. In some embodiments, the additional therapeutic agent is pegylated interferon alfa-2a, pegylated interferon alfa-2b, or a combination thereof.

In some embodiments, the additional therapeutic agent is an immunostimulatory agent. In some embodiments, the additional therapeutic agent is an oligonucleotide. In some embodiments, the additional therapeutic agent is an antimitotic inhibitor. For example, in some embodiments, the additional therapeutic agent is selected from fomivirsen, podofilox, imiquimod, sinecatechins, and combinations thereof.

In some embodiments, the additional therapeutic agent is selected from besifovir, nitazoxanide, REGN2222, doravirine, sofosbuvir, velpatasvir, daclatasvir, asunaprevir, beclabuvir, FV100, and letermovir, and combinations thereof.

In some embodiments, the additional therapeutic agent is an agent for treatment of RSV. For example, in some embodiments, the antiviral agent is ribavirin, ALS-8112 or presatovir. For example, in some embodiments, the antiviral agent is ALS-8112 or presatovir.

In some embodiments, the additional therapeutic agent is an agent for treatment of picomavirus. In some embodiments, the additional therapeutic agent is selected from hydantoin, guanidine hydrochloride, I-buthionine sulfoximine, Py-11, and combinations thereof. In some embodiments, the additional therapeutic agent is a picomavirus polymerase inhibitor. In some embodiments, the additional therapeutic agent is rupintrivir.

In some embodiments, the additional therapeutic agent is an agent for treatment of malaria. In some embodiments, the additional therapeutic agent is chloroquine.

In some embodiments, the additional therapeutic agent is selected from hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, cipargamin, artemisone, and combinations thereof.

In some embodiments, the additional therapeutic agent is an agent for treatment of coronavirus. In some embodiments, the additional therapeutic agent is selected from a group consisting of IFX-1, FM-201, CYNK-001, DPP4-Fc, ranpirnase, nafamostat, LB-2, AM-1, anti-viroporins, and combinations thereof.

In some embodiments, the additional therapeutic agent is an agent for treatment of ebola virus. For example, in some embodiments, the additional therapeutic agent is selected from ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2,3S,4R,5R)-2-(4-amino-SH-pyrrolo[3,2-d]pyrimidin-7-yl)-5-(hydroxymethyl) pyrrolidine-3,4-diol), favipiravir (also known as T-705 or Avigan), T-705 monophosphate, T-705 diphosphate, T-705 triphosphate, FGI-106 (1-N,7-N-bis [3-(dimethylamino) propyl]-3,9-dimethylquinolino [8,7-h]quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPCI inhibitors), rVSV-EBOV, and combinations thereof. In some embodiments, the additional therapeutic agent is ZMapp, mAB114, REGEN-EB3, and combinations thereof.

In some embodiments, the additional therapeutic agent is an agent for treatment of HCV. In some embodiments, the additional therapeutic agent is a HCV polymerase inhibitor. For example, in some embodiments, the additional therapeutic agent is selected from sofosbuvir, GS-6620, PSI-938, ribavirin, tegobuvir, radalbuvir, MK-0608, and combinations thereof. In some embodiments, the additional therapeutic agent is a HCV protease inhibitor. For example, in some embodiments, the additional therapeutic agent is selected from such as GS-9256, vedroprevir, voxilaprevir, and combinations thereof.

In some embodiments, the additional therapeutic agent is a NS5A inhibitor. For example, in some embodiments, the additional therapeutic agent is selected from ledipasvir, velpatasvir, and combinations thereof.

In some embodiments, the additional therapeutic agent is an anti HBV agent, For example, in some embodiments, the additional therapeutic agent is tenofovir disoproxil fumarate and emtricitabine, or a combination thereof. Examples of additional anti HBV agents include but are not limited to alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, QL-007sofosbuvir, ledipasvir, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche). In some embodiments, the additional therapeutic agent is a HBV polymerase inhibitor. Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-Nov. 4, 2026, HIP-1302, tenofovir disoproxil aspartate, tenofovir disoproxil orotate, and HS-10234. In some embodiments, the additional therapeutic agent is a HBV capsid inhibitor.

In some embodiments, the additional therapeutic agent is an agent for treatment of HIV. In some embodiments, the additional therapeutic agent is selected from HIV protease inhibitors, HIV integrase inhibitors, entry inhibitors, HIV nucleoside reverse transcriptase inhibitors, HIV nonnucleoside reverse transcriptase inhibitors, acyclic nucleoside phosphonate analogues, and combinations thereof.

In some embodiments, the additional therapeutic agent is selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies (such as chimeric antigen receptor T-cell, CAR-T, and engineered T cell receptors, TCR-T, autologous T cell therapies).

In some embodiments, the additional therapeutic agent is selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is a HIV combination drug. Examples of the HIV combination drugs include, but are not limited to ATRIPLA® (efavirenz, tenofovir disoproxil fumarate, and emtricitabine); BIKTARVY® (bictegravir, emtricitabine, and tenofovir alafenamide); COMPLERA® (EVIPLERAR; rilpivirine, tenofovir disoproxil fumarate, and emtricitabine); STRIBILD® (elvitegravir, cobicistat, tenofovir disoproxil fumarate, and emtricitabine); TRUVADAR (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIAR, lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine.

In some embodiments, the additional therapeutic agent is a HIV protease inhibitor. For example, in some embodiments the additional therapeutic agent is selected from saquinavir, ritonavir, indinavir, nelfinavir, amprenavir, lopinavir, atazanavir, fosamprenavir, darunavir, tipranavir, cobicistat, ASC-09, AEBL-2, MK-8718, GS-9500, GS-1156, and combinations thereof. For example, in some embodiments the additional therapeutic agent is selected from saquinavir, ritonavir, indinavir, nelfinavir, amprenavir, lopinavir, atazanavir, fosamprenavir, darunavir, tipranavir, cobicistat. In some embodiments, the additional therapeutic agent is selected from amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, TMC-310911, and combinations thereof.

In some embodiments, the additional therapeutic agent is a HIV integrase inhibitor. For example, in some embodiment, the additional therapeutic agent is selected from raltegravir, elvitegravir, dolutegravir, abacavir, lamivudine, bictegravir and combinations thereof. In some embodiment, the additional therapeutic agent is bictegravir. In some embodiments, the additional therapeutic agent is selected from a group consisting of bictegravir, elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, cabotegravir, and combinations thereof.

In some embodiments, the additional therapeutic agent is a HIV entry inhibitor. For example, in some embodiments, the additional therapeutic agent is selected from enfuvirtide, maraviroc, and combinations thereof. Further examples of HIV entry inhibitors include, but are not limited to, cenicriviroc, CCR5 inhibitors, gp41 inhibitors, CD4 attachment inhibitors, DS-003 (BMS-599793), gp120 inhibitors, and CXCR4 inhibitors. Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu). Examples of CXCR4 inhibitors include plerixafor, ALT-1188, N15 peptide, and vMIP (Haimipu).

In some embodiments, the additional therapeutic agent is a HIV nucleoside reverse transcriptase inhibitors. In some embodiments, the additional therapeutic agent is a HIV nonnucleoside reverse transcriptase inhibitors. In some embodiments, the additional therapeutic agent is an acyclic nucleoside phosphonate analogue. In some embodiments, the additional therapeutic agent is a HIV capsid inhibitor.

In some embodiments, the additional therapeutic agent is a HIV nucleoside or nucleotide inhibitor of reverse transcriptase. For example, the additional therapeutic agent is selected from adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500, KP-1461, and combinations thereof.

In some embodiments, the additional therapeutic agent is a HIV non-nucleoside or non-nucleotide inhibitor of reverse transcriptase. For example, the additional agent is selected from dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, elsulfavirine rilp (VM-1500), combinations thereof.

In some embodiments, the additional therapeutic agents are selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine);

TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat;
atazanavir and cobicistat; darunavir and cobicistat; atazanavir; atazanavir sulfate; dolutegravir; elvitegravir; ritonavir; atazanavir sulfate and ritonavir; darunavir; lamivudine;
prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the additional therapeutic agent is selected from colistin, valrubicin, icatibant, bepotastine, epirubicin, epoprosetnol, vapreotide, aprepitant, caspofungin, perphenazine, atazanavir, efavirenz, ritonavir, acyclovir, ganciclovir, penciclovir, prulifloxacin, bictegravir, nelfinavir, tegobuvi, nelfinavir, praziquantel, pitavastatin, perampanel, eszopiclone, and zopiclone.

In some embodiments, the additional therapeutic agent is an inhibitor of Bruton tyrosine kinase (BTK, AGMX1, AT, ATK, BPK, IGHD3, IMD1, PSCTK1, XLA; NCBI Gene ID: 695). For example, in some embodiments, the additional therapeutic agent is selected from(S)-6-amino-9-(1-(but-2-ynoyl) pyrrolidin-3-yl)-7-(4-phenoxyphenyl)-7H-purin-8 (9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, AZD6738, calquence, danvatirsen, and combinations thereof. In some embodiments, the additional therapeutic agent is selected from a group consisting of tirabrutinib, ibrutinib, acalabrutinib, and combinations thereof. In some embodiments, the additional therapeutic agent is selected from a group consisting of tirabrutinib, ibrutinib, and combinations thereof. In some embodiments, the additional therapeutic agent is tyrphostin A9 (A9).

In some embodiments, the additional therapeutic agent is a KRAS inhibitor. For example, in some embodiments, the additional therapeutic agent is selected from AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras (G12D)-selective inhibitory peptides, including KRpep-2 (Ac-RRCPLYISYDPVCRR-NH2), KRpep-2d (Ac-RRRRCPLYISYDPVCRRRR-NH2), and combinations thereof.

In some embodiments, the additional therapeutic agent is a proteasome inhibitor. For example, in some embodiments, the additional therapeutic agent is selected from a group consisting of ixazomib, carfilzomib, marizomib, bortezomib, and combinations thereof. In some embodiments, the additional therapeutic agent is carfilzomib.

In some embodiments, the additional therapeutic agent is a vaccine. For example, in some embodiments, the additional therapeutic agent is a DNA vaccine, RNA vaccine, live-attenuated vaccine, therapeutic vaccine, prophylactic vaccine, protein based vaccine, or a combination thereof. In some embodiments, the additional therapeutic agent is mRNA-1273. In some embodiments, the additional therapeutic agent is INO-4800 or INO-4700. In some embodiments, the additional therapeutic agent is live-attenuated RSV vaccine MEDI-559, human monoclonal antibody REGN2222 against RSV, palivizumab, respiratory syncytial virus immune globulin, intravenous [RSV-IGIV], and combinations thereof. In some embodiments, the additional therapeutic agent is a HBV vaccine, for example pediarix, engerix-B, and recombivax HB. In some embodiments, the additional therapeutic agent is a VZV vaccine, for example zostavax and varivax. In some embodiments, the additional therapeutic agent is a HPV vaccine, for example cervarix, gardasil 9, and gardasil. In some embodiments, the additional therapeutic agent is an influenza virus vaccine. For example, a (i) monovalent vaccine for influenza A (e.g., influenza A [H5N1] virus monovalent vaccine and influenza A [H1N1] 2009 virus monovalent vaccines), (ii) trivalent vaccine for influenza A and B viruses (e.g., Afluria, Agriflu, Fluad, Fluarix, Flublok, Flucelvax, FluLaval, Fluvirin, and Fluzone), and (iii) quadrivalent vaccine for influenza A and B viruses (FluMist, Fluarix, Fluzone, and FluLaval). In some embodiments, the additional therapeutic agent is a human adenovirus vaccine (e.g., Adenovirus Type 4 and Type 7 Vaccine, Live, Oral). In some embodiments, the additional therapeutic agent is a rotavirus vaccine (e.g., Rotarix for rotavirus serotype G1, G3, G4, or G9 and RotaTeq for rotavirus serotype G1, G2, G3, or G4). In some embodiments, the additional therapeutic agent is a hepatitis A virus vaccine (e.g., Havrix and Vaqta). In some embodiments, the additional therapeutic agent is poliovirus vaccines (e.g., Kinrix, Quadracel, and Ipol). In some embodiments, the additional therapeutic agent is a yellow fever virus vaccine (e.g., YF-Vax). In some embodiments, the additional therapeutic agent is a Japanese encephalitis virus vaccines (e.g., Ixiaro and JE-Vax). In some embodiments, the additional therapeutic agent is a measles vaccine (e.g., M-M-R II and ProQuad). In some embodiments, the additional therapeutic agent is a mumps vaccine (e.g., M-M-R II and ProQuad). In some embodiments, the additional therapeutic agent is a rubella vaccine (e.g., M-M-R II and ProQuad). In some embodiments, the additional therapeutic agent is a varicella vaccine (e.g., ProQuad). In some embodiments, the additional therapeutic agent is a rabies vaccine (e.g., Imovax and RabAvert). In some embodiments, the additional therapeutic agent is a variola virus (smallpox) vaccine (ACAM2000). In some embodiments, the additional therapeutic agent is a and hepatitis E virus (HEV) vaccine (e.g., HEV239). In some embodiments, the additional therapeutic agent is a 2019-nCov vaccine.

In some embodiments, the additional therapeutic agent is an antibody, for example a monoclonal antibody. For example, the additional therapeutic agent is an antibody against 2019-nCov selected from the Regeneron antibodies, the Wuxi Antibodies, the Vir Biotechnology Antibodies, antibodies that target the SARS-COV-2 spike protein, antibodies that can neutralize SARS-COV-2 (SARS-COV-2 neutralizing antibodies), and combinations thereof. In some embodiments, the additional therapeutic agent is anti-SARS-COV antibody CR-3022. In some embodiments, the additional therapeutic agent is aPD-1 antibody.

In some embodiments, the additional therapeutic agent is recombinant cytokine gene-derived protein injection.

In some embodiments, the additional therapeutic agent is a polymerase inhibitor. In some embodiments, the additional therapeutic agent is a DNA polymerase inhibitor. For example, in some embodiments, the additional therapeutic agent is cidofovir. In some embodiments, the additional therapeutic agent is a RNA polymerase inhibitor. For example, in some embodiments, the additional therapeutic agent is selected from ribavirin, favipiravir, lamivudine, pimodivir and combination thereof.

In some embodiments, the additional therapeutic agent is selected from lopinavir, ritonavir, interferon-alpha-2b, ritonavir, arbidol, hydroxychloroquine, darunavir and cobicistat, abidol hydrochloride, oseltamivir, litonavir, emtricitabine, tenofovir alafenamide fumarate, baloxavir marboxil, ruxolitinib, and combinations thereof.

In some embodiments, the additional therapeutic agent is selected from 6′-fluorinated aristeromycin analogues, acyclovir fleximer analogues, disulfiram, thiopurine analogues, ASC09F, GC376, GC813, phenylisoserine derivatives, neuroiminidase inhibitor analogues, pyrithiobac derivatives, bananins and 5-hydroxychromone derivatives, SSYA10-001, griffithsin, HR2P-M1, HR2P-M2, P21S10, Dihydrotanshinone E-64-C and E-64-D, OC43-HR2P, MERS-5HB, 229E-HRIP, 229E-HR2P, resveratrol, 1-thia-4-azaspiro[4.5]decan-3-one derivatives, gemcitabine hydrochloride, loperamide, recombinant interferons, cyclosporine A, alisporivir, imatinib mesylate, dasatinib, selumetinib, trametinib, rapamycin, saracatinib, chlorpromazine, triflupromazine, fluphenazine, thiethylperazine, promethazine, cyclophilin inhibitors, K11777, camostat, k22, teicoplanin derivatives, benzo-heterocyclic amine derivatives N30, mycophenolic acid, silvestrol, and combinations thereof.

In some embodiments, the additional therapeutic agent is an antibody. In some embodiments, the additional therapeutic agent is an antibody that binds to a coronavirus, for example an antibody that binds to SARS-COV or MERS-COV. In some embodiments, the additional therapeutic agent is a of 2019-nCoV virus antibody.

Compositions of the invention are also used in combination with other active ingredients. For the treatment of 2019-nCOV virus infections, preferably, the other active therapeutic agent is active against coronavirus infections, for example 2019-nCOV virus infections. The compounds and compositions of the present invention are also intended for use with general care provided patients with 2019-nCOV viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen or steroids), corticosteroids such as methylprednisolone, immonumodulatory medications (e.g., interferon), other small molecule or biologics antiviral agents targeting 2019-nCOV (such as but not limited to lopinavir/ritonavir, EIDD-1931, favipiravir, ribavirine, neutralizing antibodies, etc.), vaccines, pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cepbalosporin antibiotics, such as ceftriaxone, or aminopenicillins, such as ampicillin), or shigellosis. In some embodiments, the additional therapeutic agent is dihydroartemisinin/piperaquine. In some embodiments, the additional therapeutic agent is EIDD-2801 (MH-4482, Molnupiravir).

In some embodiments, the additional therapeutic agent is an immunomodulator. Examples of immune-based therapies include toll-like receptors modulators such as tlr1, tlr2, tlr3, tlr4, tlr5, tlr6, tlr7, tlr8, tlr9, tir10, tlr11, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, STING modulators, RIG-I modulators, NOD2 modulators, SB-9200, and IR-103. In some embodiments, the additional therapeutic agent is fingolimod, leflunomide, or a combination thereof. In some embodiments, the additional therapeutic agent is thalidomide.

In some embodiments, the additional therapeutic agent is an IL-6 inhibitor, for example tocilizumab, sarilumab, or a combination thereof.

In some embodiments, the additional therapeutic agent is an anti-TNF inhibitor. For example, the additional therapeutic agent is adalimumab, etanercept, golimumab, infliximab, or a combination thereof.

In some embodiments, the additional therapeutic agent is a JAK inhibitor, for example the additional therapeutic agent is baricitinib, filgotinib, olumiant, or a combination thereof.

In some embodiments, the additional therapeutic agent is an inflammation inhibitor, for example pirfenidone.

In some embodiments, the additional therapeutic agent is an antibiotic for secondary bacterial pneumonia. For example, the additional therapeutic agent is macrolide antibiotics (e.g., azithromycin, clarithromycin, and Mycoplasma pneumoniae), fluoroquinolones (e.g., ciprofloxacin and levofloxacin), tetracyclines (e.g., doxycycline and tetracycline), or a combination thereof.

In some embodiments, the compounds described herein are used in combination with pneumonia standard of care (see e.g., Pediatric Community Pneumonia Guidelines, CID 2011:53 (1 October)). Treatment for pneumonia generally involves curing the infection and preventing complications. Specific treatment will depend on several factors, including the type and severity of pneumonia, age and overall health of the individuals. The options include: (i) antibiotics, (ii) cough medicine, and (iii) fever reducers/pain relievers (for e.g., aspirin, ibuprofen (Advil, Motrin IB, others) and acetaminophen (Tylenol, others)). In some embodiments, the additional therapeutic agent is bromhexine anti-cough.

In some embodiments, the compounds described herein are used in combination with immunoglobulin from cured COVID-19 patients. In some embodiments, the compounds described herein are used in combination with plasma transfusion. In some embodiments, the compounds described herein are used in combination with stem cells.

In some embodiments, the additional therapeutic agent is an TLR agonist. Examples of TLR agonists include, but are not limited to, vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod, and RO-7020531.

In some embodiments, the additional therapeutic agent is selected from bortezomid, flurazepam, ponatinib, sorafenib, paramethasone, clocortolone, flucloxacillin, sertindole, clevidipine, atorvastatin, cinolazepam, clofazimine, fosaprepitant, and combinations thereof.

In some embodiments, the additional therapeutic agent is carrimycin, suramin, triazavirin, dipyridamole, bevacizumab, meplazumab, GD31 (rhizobium), NLRP inflammasome inhibitor, or a-ketoamine. In some embodiments, the additional therapeutic agent is recombinant human angiotensin-converting enzyme 2 (rhACE2). In some embodiments, the additional therapeutic agent is viral macrophage inflammatory protein (vMIP).

In some embodiments, the additional therapeutic agent is an anti-viroporin therapeutic. For example, the additional therapeutic agent is BIT-314 or BIT-225. In some embodiments, the additional therapeutic agent is coronavirus E protein inhibitor. For example, the additional therapeutic agent is BIT-009. Further examples of additional therapeutic agents include those described in WO-2004112687, WO-2006135978, WO-2018145148, and WO-2009018609.

In some embodiments, the additional therapeutic or prophylactic agent is molnupiravir, oseltamivir, nirmatrelvir, or ritonavir. In some embodiments, the additional therapeutic or prophylactic agent is ritonavir or cobicistat.

In some embodiments, the additional therapeutic agent a 2,5-Oligoadenylate synthetase stimulator, 5-HT 2a receptor antagonist, 5-Lipoxygenase inhibitor, ABL family tyrosine kinase inhibitor, Abl tyrosine kinase inhibitor, Acetaldehyde dehydrogenase inhibitor, Acetyl CoA carboxylase inhibitor, Actin antagonist, Actin modulator, Activity-dependent neuroprotector modulator, Adenosine A3 receptor agonist, Adrenergic receptor antagonist, Adrenomedullin ligand, Adrenomedullin ligand inhibitor, Advanced glycosylation product receptor antagonist, Advanced glycosylation product receptor modulator, AKT protein kinase inhibitor, Alanine proline rich secreted protein stimulator, Aldose reductase inhibitor, Alkaline phosphatase stimulator, Alpha 2 adrenoceptor antagonist, Alpha 2B adrenoceptor agonist, AMP activated protein kinase stimulator, AMPA receptor modulator, Amyloid protein deposition inhibitor, Androgen receptor antagonist, Angiotensin II AT-I receptor antagonist, Angiotensin II AT-2 receptor agonist, Angiotensin II receptor modulator, Angiotensin converting enzyme 2 inhibitor, Angiotensin converting enzyme 2 modulator, Angiotensin converting enzyme 2 stimulator, Angiotensin receptor modulator, Annexin AS stimulator, Anoctamin 1 inhibitor, Anti-coagulant, Anti-histamine, Anti-hypoxic, Anti-thrombotic, AP1 transcription factor modulator, Apelin receptor agonist, APOA1 gene stimulator, Apolipoprotein A1 agonist, Apolipoprotein B antagonist, Apolipoprotein B modulator, Apolipoprotein C3 antagonist, Aryl hydrocarbon receptor agonist, Aryl hydrocarbon receptor antagonist, ATP binding cassette transporter B5 modulator, Axl tyrosine kinase receptor inhibitor, Bactericidal permeability protein inhibitor, Basigin inhibitor, Basigin modulator, BCL2 gene inhibitor, BCL2L11 gene stimulator, Bcr protein inhibitor, Beta 1 adrenoceptor modulator, Beta 2 adrenoceptor agonist, Beta adrenoceptor agonist, Beta-arrestin stimulator, Blood clotting modulator, BMP10 gene inhibitor, BMP15 gene inhibitor, Bone morphogenetic protein-10 ligand inhibitor, Bone morphogenetic protein-15 ligand inhibitor, Bradykinin B2 receptor antagonist, Brain derived neurotrophic factor ligand, Bromodomain containing protein 2 inhibitor, Bromodomain containing protein 4 inhibitor, Btk tyrosine kinase inhibitor, C-reactive protein modulator, Ca2+release activated Ca2+channel 1 inhibitor, Cadherin-5 modulator, Calcium activated chloride channel inhibitor, Calcium channel modulator, Calpain-I inhibitor, Calpain-II inhibitor, Calpain-IX inhibitor, Cannabinoid CB2 receptor agonist, Cannabinoid receptor modulator, Casein kinase II inhibitor, CASP8-FADD-like regulator inhibitor, Caspase inhibitor, Catalase stimulator, CCL26 gene inhibitor, CCR2 chemokine antagonist, CCR5 chemokine antagonist, CD11a agonist, CD122 agonist, CD3 antagonist, CD4 agonist, CD40 ligand, CD40 ligand modulator, CD40 ligand receptor agonist, CD40 ligand receptor modulator, CD49d agonist, CD70 antigen modulator, CD73 agonist, CD73 antagonist, CD95 antagonist, CFTR inhibitor, CGRP receptor antagonist, Chemokine receptor-like 1 agonist, Chloride channel inhibitor, Chloride channel modulator, Cholera enterotoxin subunit B inhibitor, Cholesterol ester transfer protein inhibitor, Collagen modulator, Complement C1s subcomponent inhibitor, Complement C3 inhibitor, Complement C5 factor inhibitor, Complement C5a factor inhibitor, Complement Factor H stimulator, Complement cascade inhibitor, Complement factor C2 inhibitor, Complement factor D inhibitor, Connective tissue growth factor ligand inhibitor, Coronavirus nucleoprotein modulator, Coronavirus small envelope protein modulator, Coronavirus spike glycoprotein inhibitor, Coronavirus spike glycoprotein modulator, COVID19 envelope small membrane protein modulator, COVID19 nonstructural protein 8 modulator, COVID19 nucleoprotein modulator, COVID19 Protein 3a inhibitor, COVID19 replicase polyprotein 1a inhibitor, COVID19 replicase polyprotein 1a modulator, COVID19 replicase polyprotein lab inhibitor, COVID19 replicase polyprotein lab modulator, COVID19 Spike glycoprotein inhibitor, COVID19 Spike glycoprotein modulator, COVID19 structural glycoprotein modulator, CRF-2 receptor agonist, CSF-1 agonist, CSF-1 antagonist, CX3CR1 chemokine antagonist, CXC10 chemokine ligand inhibitor, CXC5 chemokine ligand inhibitor, CXCL1 gene modulator, CXCL2 gene modulator, CXCL3 gene modulator, CXCR1 chemokine antagonist, CXCR2 chemokine antagonist, CXCR4 chemokine antagonist, Cyclin D1 inhibitor, Cyclin E inhibitor, Cyclin-dependent kinase-1 inhibitor, Cyclin-dependent kinase-2 inhibitor, Cyclin-dependent kinase-5 inhibitor, Cyclin-dependent kinase-7 inhibitor, Cyclin-dependent kinase-9 inhibitor, Cyclooxygenase 2 inhibitor, Cyclooxygenase inhibitor, Cyclophilin inhibitor, Cysteine protease inhibitor, Cytochrome P450 3A4 inhibitor, Cytokine receptor antagonist, Cytotoxic T lymphocyte protein gene modulator, Cytotoxic T-lymphocyte protein-4 inhibitor, Cytotoxic T-lymphocyte protein-4 stimulator, DDX3 inhibitor, Dehydrogenase inhibitor, Dehydropeptidase-1 modulator, Deoxyribonuclease I stimulator, Deoxyribonuclease gamma stimulator, Deoxyribonuclease stimulator, Dihydroceramide delta 4 desaturase inhibitor, Dihydroorotate dehydrogenase inhibitor, Dipeptidyl peptidase I inhibitor, Dipeptidyl peptidase III inhibitor, Diuretic, DNA binding protein inhibitor, DNA methyltransferase inhibitor, Dopamine transporter inhibitor, E selectin antagonist, Ecto NOX disulfide thiol exchanger 2 inhibitor, EGFR gene inhibitor, Elongation factor 1 alpha 2 modulator, Endoplasmin modulator, Endoribonuclease DICER modulator, Endothelin ET-A receptor antagonist, Epidermal growth factor receptor antagonist, E-selectin antagonist, Estrogen receptor beta agonist, Estrogen receptor modulator, Eukaryotic initiation factor 4A-I inhibitor, Exo-alpha sialidase modulator, Exportin 1 inhibitor, Factor Ia modulator, Factor IIa modulator, Factor VII antagonist, Factor Xa antagonist, Factor XIa antagonist, FGF receptor antagonist, FGF-1 ligand, FGF-1 ligand inhibitor, FGF-2 ligand inhibitor, FGF1 receptor antagonist, FGF2 receptor antagonist, FGF3 receptor antagonist, Flt3 tyrosine kinase inhibitor, Fractalkine ligand inhibitor, Free fatty acid receptor 2 agonist, Free fatty acid receptor 3 agonist, furin inhibitors, Fyn tyrosine kinase inhibitor, FYVE finger phosphoinositide kinase inhibitor, G-protein coupled bile acid receptor 1 agonist, GABA A receptor modulator, Galectin-3 inhibitor, Gamma-secretase inhibitor, GDF agonist, Gelsolin stimulator, Glial cell neurotrophic factor ligand, Glucocorticoid receptor agonist, Glutathione peroxidase stimulator, GM-CSF ligand inhibitor, GM-CSF receptor agonist, GM-CSF receptor modulator, Griffithsin modulator, Growth regulated protein alpha ligand inhibitor, Grp78 calcium binding protein inhibitor, Heat shock protein HSP90 alpha inhibitor, Heat shock protein HSP90 beta inhibitor, Heat shock protein inhibitor, Heat shock protein stimulator, Hemagglutinin modulator, Hemoglobin modulator, Hemolysin alpha inhibitor, Heparanase inhibitor, Heparin agonist, Hepatitis B structural protein inhibitor, Hepatitis C virus NS5B polymerase inhibitor, HIF prolyl hydroxylase inhibitor, HIF prolyl hydroxylase-2 inhibitor, High mobility group protein B1 inhibitor, Histamine H1 receptor antagonist, Histamine H2 receptor antagonist, Histone deacetylase-6 inhibitor, Histone inhibitor, HIV protease inhibitor, HIV-1 gp120 protein inhibitor, HIV-1 protease inhibitor, HIV-1 reverse transcriptase inhibitor, HLA class I antigen modulator, HLA class II antigen modulator, Host cell factor modulator, Hsp 90 inhibitor, Human papillomavirus E6 protein modulator, Human papillomavirus E7 protein modulator, Hypoxia inducible factor inhibitor gene inhibitor, Hypoxia inducible factor-2 alpha modulator, I-kappa B kinase inhibitor, I-kappa B kinase modulator, ICAM-1 stimulator, IgG receptor FcRn large subunit p51 modulator, IL-12 receptor antagonist, IL-15 receptor agonist, IL-15 receptor modulator, IL-17 antagonist, IL-18 receptor accessory protein antagonist, IL-2 receptor agonist, IL-22 agonist, IL-23 antagonist, IL-6 receptor agonist, IL-6 receptor antagonist, IL-6 receptor modulator, IL-7 receptor agonist, IL-8 receptor antagonist, IL12 gene stimulator, IL8 gene modulator, Immunoglobulin G modulator, Immunoglobulin G1 agonist, Immunoglobulin G1 modulator, Immunoglobulin agonist, Immunoglobulin gamma Fc receptor I modulator, Immunoglobulin kappa modulator, Inosine monophosphate dehydrogenase inhibitor, Insulin sensitizer, Integrin agonist, Integrin alpha-4/beta-7 antagonist, Integrin alpha-V/beta-1 antagonist, Integrin alpha-V/beta-6 antagonist, Interferon agonist, Interferon alpha 14 ligand, Interferon alpha 2 ligand, Interferon alpha 2 ligand modulator, Interferon alpha ligand, Interferon alpha ligand inhibitor, Interferon alpha ligand modulator, Interferon beta ligand, Interferon gamma ligand inhibitor, Interferon gamma receptor agonist, Interferon gamma receptor antagonist, Interferon receptor modulator, Interferon type I receptor agonist, Interleukin 17A ligand inhibitor, Interleukin 17F ligand inhibitor, Interleukin 18 ligand inhibitor, Interleukin 22 ligand, Interleukin-1 beta ligand inhibitor, Interleukin-1 beta ligand modulator, Interleukin-1 ligand inhibitor, Interleukin-2 ligand, Interleukin-29 ligand, Interleukin-6 ligand inhibitor, Interleukin-7 ligand, Interleukin-8 ligand inhibitor, IRAK-4 protein kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Jun N terminal kinase modulator, Kallikrein modulator, Kelch like ECH associated protein 1 modulator, Kit tyrosine kinase inhibitor, KLKB1 gene inhibitor, Lactoferrin stimulator, Lanosterol-14 demethylase inhibitor, Lek tyrosine kinase inhibitor, Leukocyte Ig like receptor A4 modulator, Leukocyte elastase inhibitor, Leukotriene BLT receptor antagonist, Leukotriene D4 antagonist, Leukotriene receptor antagonist, Listeriolysin stimulator, Liver X receptor antagonist, Low molecular weight heparin, Lung surfactant associated protein B stimulator, Lung surfactant associated protein D modulator, Lyn tyrosine kinase inhibitor, Lyn tyrosine kinase stimulator, Lysine specific histone demethylase 1 inhibitor, Macrophage migration inhibitory factor inhibitor, Mannan-binding lectin serine protease inhibitor, Mannan-binding lectin serine protease-2 inhibitor, MAO B inhibitor, MAP kinase inhibitor, MAPK gene modulator, Matrix metalloprotease modulator, Maxi K potassium channel inhibitor, MCLI gene inhibitor, MEK protein kinase inhibitor, MEK-1 protein kinase inhibitor, Melanocortin MC1 receptor agonist, Melanocortin MC3 receptor agonist, Metalloprotease-12 inhibitor, METTL3 gene inhibitor, Moesin inhibitor, Moesin modulator, Monocyte chemotactic protein 1 ligand inhibitor, Monocyte differentiation antigen CD14 inhibitor, mRNA cap guanine N7 methyltransferase modulator, mTOR complex 1 inhibitor, mTOR complex 2 inhibitor, mTOR inhibitor, Mucolipin modulator, Muscarinic receptor antagonist, Myeloperoxidase inhibitor, NACHT LRR PYD domain protein 3 inhibitor, NAD synthase modulator, NADPH oxidase inhibitor, Neuropilin 2 modulator, Neuroplastin inhibitor, NFE2L2 gene stimulator, NK cell receptor agonist, NKI receptor antagonist, NMDA receptor antagonist, NMDA receptor epsilon 2 subunit inhibitor, Non receptor tyrosine kinase TYK2 antagonist, Non-nucleoside reverse transcriptase inhibitor, nsp12 polymerase inhibitor, Nuclear erythroid 2-related factor 2 stimulator, Nuclear factor kappa B inhibitor, Nuclear factor kappa B modulator, Nuclease stimulator, Nucleolin inhibitor, Nucleoprotein inhibitor, Nucleoprotein modulator, Nucleoside reverse transcriptase inhibitor, Opioid receptor agonist, Opioid receptor antagonist, Opioid receptor mu modulator, Opioid receptor sigma antagonist 1, ORFlab polyprotein inhibitor, Omithine decarboxylase inhibitor, Outer membrane protein inhibitor, OX40 ligand, p38 MAP kinase alpha inhibitor, p38 MAP kinase inhibitor, p38 MAP kinase modulator, p53 tumor suppressor protein stimulator, Palmitoyl protein thioesterase 1 inhibitor, Papain inhibitor, PARP inhibitor, PARP modulator, PDE 10 inhibitor, PDE 3 inhibitor, PDE 4 inhibitor, PDGF receptor alpha antagonist, PDGF receptor antagonist, PDGF receptor beta antagonist, Peptidyl-prolyl cis-trans isomerase A inhibitor, Peroxiredoxin 6 modulator, PGD2 antagonist, PGI2 agonist, P-glycoprotein inhibitor, Phosphoinositide 3-kinase inhibitor, Phosphoinositide-3 kinase delta inhibitor, Phosphoinositide-3 kinase gamma inhibitor, Phospholipase A2 inhibitor, PIKfyve inhibitor, Plasma kallikrein inhibitor, Plasminogen activator inhibitor 1 inhibitor, Platelet inhibitor, Platelet glycoprotein VI inhibitor, Polo-like kinase 1 inhibitor, Poly ADP ribose polymerase 1 inhibitor, Poly ADP ribose polymerase 2 inhibitor, Polymerase cofactor VP35 inhibitor, PPAR alpha agonist, Progesterone receptor agonist, Programmed cell death protein 1 modulator, Prolyl hydroxylase inhibitor, Prostaglandin E synthase-1 inhibitor, Protease inhibitor, Proteasome inhibitor, Protein arginine deiminase IV inhibitor, Protein tyrosine kinase inhibitor, Protein tyrosine phosphatase beta inhibitor, Protein tyrosine phosphatase-2C inhibitor, Proto-oncogene Mas agonist, Purinoceptor antagonist, Raf protein kinase inhibitor, RANTES ligand, Ras gene inhibitor, Retinoate receptor responder protein 2 stimulator, Rev protein modulator, Ribonuclease stimulator, RIP-1 kinase inhibitor, RNA helicase inhibitor, RNA polymerase inhibitor, RNA polymerase modulator, S phase kinase associated protein 2 inhibitor, SARS coronavirus 3C protease like inhibitor, Serine protease inhibitor, Serine threonine protein kinase ATR inhibitor, Serine threonine protein kinase TBK1 inhibitor, Serum amyloid A protein modulator, Signal transducer CD24 stimulator, Sirtuin inhibitor, Sodium channel stimulator, Sodium glucose transporter-2 inhibitor, Sphingosine kinase 1 inhibitor, Sphingosine kinase 2 inhibitor, Sphingosine kinase inhibitor, Sphingosine-1-phosphate receptor-1 agonist, Sphingosine-1-phosphate receptor-1 antagonist, Sphingosine-1-phosphate receptor-1 modulator, Sphingosine-1-phosphate receptor-5 agonist, Sphingosine-1-phosphate receptor-5 modulator, Spike glycoprotein inhibitor, Src tyrosine kinase inhibitor, STAT-1 modulator, STAT-3 inhibitor, STAT-5 inhibitor, STAT3 gene inhibitor, Stem cell antigen-1 inhibitor, Stimulator of interferon genes protein stimulator, Sulfatase inhibitor, Superoxide dismutase modulator, Superoxide dismutase stimulator, Syk tyrosine kinase inhibitor, T cell immunoreceptor Ig ITIM protein inhibitor, T cell receptor agonist, T cell surface glycoprotein CD28 inhibitor, T-cell differentiation antigen CD6 inhibitor, T-cell surface glycoprotein CD8 stimulator, T-cell transcription factor NFAT modulator, Tankyrase-1 inhibitor, Tankyrase-2 inhibitor, Tek tyrosine kinase receptor stimulator, Telomerase modulator, Tetanus toxin modulator, TGF beta receptor antagonist, TGFB2 gene inhibitor, Thymosin beta 4 ligand, Thyroid hormone receptor beta agonist, Tissue factor inhibitor, Tissue plasminogen activator modulator, Tissue plasminogen activator stimulator, TLR agonist, TLR modulator, TLR-2 agonist, TLR-2 antagonist, TLR-3 agonist, TLR-4 agonist, TLR-4 antagonist, TLR-6 agonist, TLR-7 agonist, TLR-7 antagonist, TLR-8 antagonist, TLR-9 agonist, TMPRSS2 gene inhibitor, TNF alpha ligand inhibitor, TNF alpha ligand modulator, TNF binding agent, TNF gene inhibitor, Topoisomerase inhibitor, Transcription factor EB stimulator, Transferrin modulator, Transketolase inhibitor, Translocation associated protein inhibitor, Transmembrane serine protease 2 inhibitor, Transthyretin modulator, TREM receptor 1 antagonist, TRP cation channel CI modulator, TRP cation channel C6 inhibitor, TRP cation channel V6 inhibitor, Trypsin 1 inhibitor, Trypsin 2 inhibitor, Trypsin 3 inhibitor, Trypsin inhibitor, Tubulin alpha inhibitor, Tubulin beta inhibitor, Tumor necrosis factor 14 ligand inhibitor, TYK2 gene inhibitor, Type I IL-1 receptor antagonist, Tyrosine protein kinase ABL1 inhibitor, Ubiquinol cytochrome C reductase 14 kDa inhibitor, Ubiquitin ligase modulator, Unspecified GPCR agonist, Unspecified cytokine receptor modulator, Unspecified enzyme stimulator, Unspecified gene inhibitor, Unspecified receptor modulator, Urokinase plasminogen activator inhibitor, Vascular cell adhesion protein 1 agonist, Vasodilator, VEGF ligand inhibitor, VEGF receptor antagonist, VEGF-1 receptor antagonist, VEGF-1 receptor modulator, VEGF-2 receptor antagonist, VEGF-3 receptor antagonist, Vimentin inhibitor, Vimentin modulator, VIP receptor agonist, Viral envelope protein inhibitor, Viral protease inhibitor, Viral protease modulator, Viral protein target modulator, Viral ribonuclease inhibitor, Viral structural protein modulator, Vitamin D3 receptor agonist, X-linked inhibitor of apoptosis protein inhibitor, Xanthine oxidase inhibitor, or Zonulin inhibitor.

In some embodiments, the compounds and compositions of the present disclosure may be administered in combination with a Sars-Cov-2 treatment, such as parenteral fluids (including dextrose saline and Ringer's lactate), nutrition, antibiotics (including azithromycin, metronidazole, amphotericin B, amoxicillin/clavulanate, trimethoprim/sulfamethoxazole, R-327 and cephalosporin antibiotics, such as ceftriaxone and cefuroxime), antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as dexamethasone, methylprednisolone, prednisone, mometasone, immunomodulatory medications (eg interferon), vaccines, and pain medications.

In some embodiments, the additional therapeutic agent is an Abl tyrosine kinase inhibitor, such as radotinib or imatinib.

In some embodiments, the additional therapeutic agent is an acetaldehyde dehydrogenase inhibitor, such as ADX-629.

In some embodiments, the additional therapeutic agent is an adenosine A3 receptor agonist, such as piclidenoson.

In some embodiments, the additional therapeutic agent is an adrenomedullin ligand such as adrenomedullin.

In some embodiments, the additional therapeutic agent is a p38 MAPK+PPAR gamma agonist/insulin sensitizer such as KIN-001.

In some embodiments, the additional therapeutic agent is a PPAR alpha agonist such as DWTC-5101 (fenofibrate choline).

In some embodiments, the additional therapeutic agent is a cyclophilin inhibitor such as rencofilstat.

In some embodiments, the additional therapeutic is a p38 MAP kinase inhibitor such as PRX-201 or Gen-1124.

In some embodiments, the additional therapeutic agent is an aldose reductase inhibitor, such as caficrestat.

In some embodiments, the additional therapeutic agent is an AMPA receptor modulator, such as traneurocin.

In some embodiments, the additional therapeutic agent is an annexin A5 stimulator, such as AP-01 or SY-005.

In some embodiments, the additional therapeutic agent is an apelin receptor agonist, such as CB-5064 MM.

In some embodiments, the additional therapeutic agent is an anti-coagulant, such as heparins (heparin and low molecular weight heparin), aspirin, apixaban, dabigatran, edoxaban, argatroban, enoxaparin, or fondaparinux.

In some embodiments, the additional therapeutic agent is an androgen receptor antagonist such as bicalutamide, deutenzalutamide, enzalutamide, or pruxelutamide (proxalutamide)

In some embodiments, the additional therapeutic agent is anti-hypoxic, such as trans-sodium crocetinate.

In some embodiments, the additional therapeutic agent is an anti-thrombotic, such as defibrotide, rivaroxaban, alteplase, tirofiban, clopidogrel, prasugrel, bemiparin, bivalirudin, sulodexide, or tenecteplase.

In some embodiments, the additional therapeutic agent is an antihistamine, such as cloroperastine or clemastine.

In some embodiments, the additional therapeutic agent is an apolipoprotein A1 agonist, such as CER-001.

In some embodiments, the additional therapeutic agent is a phospholipase A2 inhibitor, such as icosapent ethyl.

In some embodiments, the additional therapeutic agent is an axl tyrosine kinase receptor inhibitor, such as bemcentinib.

In some embodiments, the additional therapeutic agent is a corticosteroid/beta 2 adrenoceptor agonist, such as budesonide+formoterol fumarate.

In some embodiments, the additional therapeutic agent is a BET bromodomain inhibitor/APOA1 gene stimulator such as apabetalone.

In some embodiments, the additional therapeutic agent is a blood clotting modulator, such as lanadelumab.

In some embodiments, the additional therapeutic agent is a bradykinin B2 receptor antagonist, such as icatibant.

In some embodiments, the additional therapeutic agent is an EGFR gene inhibitor/Btk tyrosine kinase inhibitor, such as abivertinib.

In some embodiments, the additional therapeutic agent is a Btk tyrosine kinase inhibitor, such as ibrutinib or zanubrutinib.

In some embodiments, the additional therapeutic agent is a calpain-I/II/IX inhibitor, such as BLD-2660.

In some embodiments, the additional therapeutic agent is a cannabinoid CB2 receptor agonist, such as onternabez or PPP-003.

In some embodiments, the additional therapeutic agent is a Ca2+release activated Ca2+channel 1 inhibitor, such as zegocractin (CM-4620).

In some embodiments, the additional therapeutic agent is an ATR inhibitor, such as berzosertib.

In some embodiments, the additional therapeutic agent is a cadherin-5 modulator, such as FX-06.

In some embodiments, the additional therapeutic agent is a casein kinase II inhibitor, such as silmitasertib.

In some embodiments, the additional therapeutic agent is a caspase inhibitor, such as emricasan.

In some embodiments, the additional therapeutic agent is a catalase stimulator/superoxide dismutase stimulator, such as MP-1032.

In some embodiments, the additional therapeutic agent is a CCR2 chemokine antagonist/CCR5 chemokine antagonist such as cenicriviroc.

In some embodiments, the additional therapeutic agent is a CCR5 chemokine antagonist, such as maraviroc or leronlimab.

In some embodiments, the additional therapeutic agent is a CD122 agonist/IL-2 receptor agonist, such as bempegaldesleukin.

In some embodiments, the additional therapeutic agent is a CD73 agonist/interferon beta ligand, such as FP-1201.

In some embodiments, the additional therapeutic agent is a cholesterol ester transfer protein inhibitor, such as dalcetrapib.

In some embodiments, the additional therapeutic agent is a Mannan-binding lectin serine protease/complement C1s subcomponent inhibitor/myeloperoxidase inhibitor, such as RLS-0071.

In some embodiments, the additional therapeutic agent is a complement C5 factor inhibitor/leukotriene BLT receptor antagonist, such as nomacopan.

In some embodiments, the additional therapeutic agent is a complement C5 factor inhibitor, such as eculizumab, STSA-1002, zilucoplan.

In some embodiments, the additional therapeutic agent is a CXCR4 chemokine antagonist, such as plerixafor or motixafortide.

In some embodiments, the additional therapeutic agent is a cytochrome P450 3A4 inhibitor/peptidyl-prolyl cis-trans isomerase A inhibitor, such as alisporivir.

In some embodiments, the additional therapeutic agent is a cysteine protease inhibitor, such as SLV-213.

In some embodiments, the additional therapeutic agent is a dihydroorotate dehydrogenase inhibitor, such as Meds-433, brequinar, RP-7214, farudostat or emvododstat.

In some embodiments, the additional therapeutic agent is a dehydropeptidase-1 modulator, such as Metablok.

In some embodiments, the additional therapeutic agent is a dihydroorotate dehydrogenase inhibitor/IL-17 antagonist, such as vidofludimus.

In some embodiments, the additional therapeutic agent is a diuretic, such as an aldosterone antagonist, such as spironolactone.

In some embodiments, the additional therapeutic agent is a deoxyribonuclease I stimulator, such as GNR-039 or dornase alfa.

In some embodiments, the additional therapeutic agent is a NET inhibitor, such as NTR-441.

In some embodiments, the additional therapeutic agent is a dihydroceramide delta 4 desaturase inhibitor/sphingosine kinase 2 inhibitor, such as opaganib.

In some embodiments, the additional therapeutic agent is a DNA methyltransferase inhibitor, such as azacytidine.

In some embodiments, the additional therapeutic agent is an LXR antagonist, such as larsucosterol.

In some embodiments, the additional therapeutic agent is a dipeptidyl peptidase I inhibitor, such as brensocatib.

In some embodiments, the additional therapeutic agent is a protein arginine deiminase IV inhibitor, such as JBI-1044.

In some embodiments, the additional therapeutic agent is an elongation factor 1 alpha 2 modulator, such as plitidepsin.

In some embodiments, the additional therapeutic agent is a eukaryotic initiation factor 4A-I inhibitor, such as zotatifin.

In some embodiments, the additional therapeutic agent is an exo-alpha sialidase modulator, such as DAS-181.

In some embodiments, the additional therapeutic agent is an exportin 1 inhibitor, such as selinexor.

In some embodiments, the additional therapeutic agent is a fractalkine ligand inhibitor, such as KAND-567.

In some embodiments, the additional therapeutic agent is a FYVE finger phosphoinositide kinase inhibitor/IL-12 receptor antagonist/IL-23 antagonist, such as apilimod dimesylate.

In some embodiments, the additional therapeutic agent is a GABA A receptor modulator, such as brexanolone.

In some embodiments, the additional therapeutic agent is a glucocorticoid receptor agonist, such as ciclesonide, hydrocortisone, dexamethasone, dexamethasone phosphate, or 101-PGC-005.

In some embodiments, the additional therapeutic agent is a GM-CSF receptor agonist, such as sargramostim.

In some embodiments, the additional therapeutic agent is a GPCR agonist, such as esuberaprost sodium.

In some embodiments, the additional therapeutic agent is a Griffithsin modulator, such as Q-Griffithsin.

In some embodiments, the additional therapeutic agent is a leukotriene D4 antagonist, such as montelukast

In some embodiments, the additional therapeutic agent is a histamine H1 receptor antagonist, such as ebastine, tranilast, levocetirizine dihydrochloride.

In some embodiments, the additional therapeutic agent is a histamine H2 receptor antagonist, such as famotidine.

In some embodiments, the additional therapeutic agent is a heat shock protein stimulator/insulin sensitizer/PARP inhibitor, such as BGP-15.

In some embodiments, the additional therapeutic agent is a histone inhibitor, such as STC-3141.

In some embodiments, the additional therapeutic agent is a histone deacetylase-6 inhibitor, such as CKD-506.

In some embodiments, the additional therapeutic agent is a HIF prolyl hydroxylase-2 inhibitor, such as desidustat.

In some embodiments, the additional therapeutic agent is an HIF prolyl hydroxylase inhibitor, such as vadadustat.

In some embodiments, the additional therapeutic agent is an IL-8 receptor antagonist, such as reparixin.

In some embodiments, the additional therapeutic agent is an IL-7 receptor agonist, such as CYT-107.

In some embodiments, the additional therapeutic agent is an IL-7 receptor agonist/interleukin-7 ligand, such as efineptakin alfa.

In some embodiments, the additional therapeutic agent is an IL-22 agonist, such as efmarodocokin alfa.

In some embodiments, the additional therapeutic agent is an IL-22 agonist/interleukin 22 ligand, such as F-652.

In some embodiments, the additional therapeutic agent is targeted to IL-33, such as tozorakimab.

In some embodiments, the additional therapeutic is an IL-15 agonist such as nogapendekin alfa.

In some embodiments, the additional therapeutic agent is an integrin alpha-V/beta-1 antagonist/integrin alpha-V/beta-6 antagonist, such as bexotegrast.

In some embodiments, the additional therapeutic agent is an interferon alpha 2 ligand, such as interferon alfa-2b or Virafin.

In some embodiments, the additional therapeutic agent is an interferon beta ligand, such as interferon beta-1a follow-on biologic, interferon beta-1b, or SNG-001.

In some embodiments, the additional therapeutic agent is an interferon receptor modulator, such as peginterferon lambda-1a.

In some embodiments, the additional therapeutic agent is an interleukin-2 ligand, such as aldesleukin.

In some embodiments, the additional therapeutic agent is an IRAK-4 protein kinase inhibitor, such as zimlovisertib.

In some embodiments, the additional therapeutic agent is a JAK inhibitor, for example the additional therapeutic agent is baricitinib, filgotinib, jaktinib, tofacitinib, or nezulcitinib (TD-0903).

In some embodiments, the additional therapeutic agent is a neutrophil elastase inhibitor, such as alvelestat.

In some embodiments, the additional therapeutic agent is a lung surfactant associated protein D modulator, such as AT-100.

In some embodiments, the additional therapeutic agent is a plasma kallikrein inhibitor, such as donidalorsen.

In some embodiments, the additional therapeutic agent is a lysine specific histone demethylase I/MAO B inhibitor, such as vafidemstat.

In some embodiments, the additional therapeutic agent is a Mannan-binding lectin serine protease inhibitor, such as conestat alfa.

In some embodiments, the additional therapeutic agent is a maxi K potassium channel inhibitor, such as ENA-001.

In some embodiments, the additional therapeutic agent is a MEK protein kinase inhibitor, such as zapnometinib.

In some embodiments, the additional therapeutic agent is a MEK-1 protein kinase inhibitor/Ras gene inhibitor, such as antroquinonol.

In some embodiments, the additional therapeutic agent is a melanocortin MC1 receptor agonist, such as PL-8177

In some embodiments, the additional therapeutic agent is a melanocortin MC1/MC3 receptor agonist, such as resomelagon acetate.

In some embodiments, the additional therapeutic agent is a matrix metalloprotease-12 inhibitor, such as FP-025.

In some embodiments, the additional therapeutic agent is a NACHT LRR PYD domain protein 3 inhibitor, such as dapansutrile, DFV-890, or ZYIL-1.

In some embodiments, the additional therapeutic agent is a NADPH oxidase inhibitor, such as isuzinaxib.

In some embodiments, the additional therapeutic agent is a neuropilin 2 modulator, such as efzofitimod.

In some embodiments, the additional therapeutic agent is an NKI receptor antagonist, such as aprepitant or tradipitant.

In some embodiments, the additional therapeutic agent is an NMDA receptor antagonist, such as transcrocetin or ifenprodil.

In some embodiments, the additional therapeutic agent is a nuclear factor kappa B inhibitor/p38 MAP kinase inhibitor, such as zenuzolac.

In some embodiments, the additional therapeutic agent is an ornithine decarboxylase inhibitor, such as eflorithine.

In some embodiments, the additional therapeutic agent is an opioid receptor sigma antagonist 1, such as MR-309.

In some embodiments, the additional therapeutic agent is a PGD2 antagonist, such as asapiprant.

In some embodiments, the additional therapeutic agent is a PDGF receptor antagonist/TGF beta receptor antagonist/p38 MAP kinase inhibitor, such as deupirfenidone.

In some embodiments, the additional therapeutic agent is a phospholipase A2 inhibitor, such as varespladib methyl.

In some embodiments, the additional therapeutic agent is a phosphoinositide 3-kinase inhibitor/mTOR complex inhibitor, such as dactolisib.

In some embodiments, the additional therapeutic agent is a mTOR inhibitor, such as sirolimus.

In some embodiments, the additional therapeutic agent is a phosphoinositide-3 kinase delta/gamma inhibitor, such as duvelisib.

In some embodiments, the additional therapeutic agent is a PIKfyve inhibitor, such as VRG-101.

In some embodiments, the additional therapeutic agent is a plasminogen activator inhibitor 1 inhibitor, such as TM-5614.

In some embodiments, the additional therapeutic agent is a protein tyrosine phosphatase beta inhibitor, such as razuprotafib.

In some embodiments, the additional therapeutic agent is a RIP-1 kinase inhibitor, such as eclitasertib (DNL-758) or SIR-0365.

In some embodiments, the additional therapeutic agent is a Rev protein modulator, such as obefazimod.

In some embodiments, the additional therapeutic agent is an S phase kinase associated protein 2 inhibitor, such as niclosamide, CP-COV3, SCAI-502 or DWRX-2003.

In some embodiments, the additional therapeutic agent is a signal transducer CD24 stimulator, such as EXO-CD24.

In some embodiments, the additional therapeutic agent is a sodium glucose transporter-2 inhibitor, such as dapagliflozin propanediol.

In some embodiments, the additional therapeutic agent is a sodium channel stimulator, such as solnatide.

In some embodiments, the additional therapeutic agent is a sphingosine-1-phosphate receptor-1 agonist/sphingosine-1-phosphate receptor-5 agonist, such as ozanimod.

In some embodiments, the additional therapeutic agent is a non-steroidal anti-inflammatory drug, such as Ampion.

In some embodiments, the additional therapeutic agent is a superoxide dismutase stimulator, such as avasopasem manganese.

In some embodiments, the additional therapeutic agent is a Syk tyrosine kinase inhibitor, such as fostamatinib disodium.

In some embodiments, the additional therapeutic agent is a Tie2 tyrosine kinase receptor agonist, such as AV-001.

In some embodiments, the additional therapeutic agent is a TGFB2 gene inhibitor, such as trabedersen.

In some embodiments, the additional therapeutic agent is a tissue factor inhibitor, such as AB-201.

In some embodiments, the additional therapeutic agent is a TLR-3 agonist, such as rintatolimod.

In some embodiments, the additional therapeutic agent is a TLR-4 antagonist, such as ApTLR-4 FT, EB-05, or eritoran.

In some embodiments, the additional therapeutic agent is a TLR-7/8 antagonist, such as enpatoran.

In some embodiments, the additional therapeutic agent is a TLR-2/6 agonist, such as INNA-051.

In some embodiments, the additional therapeutic agent is a TLR-7 agonist, such as PRTX-007 or APR-002.

In some embodiments, the additional therapeutic agent is a TLR agonist, such as PUL-042.

In some embodiments, the additional therapeutic agent is a TLR-4 agonist, such as REVTx-99.

In some embodiments, the additional therapeutic agent is a TLR-2/4 antagonist, such as VB-201.

In some embodiments, the additional therapeutic agent is a TNF alpha ligand inhibitor, such as pegipanermin.

In some embodiments, the additional therapeutic agent is a type I IL-1 receptor antagonist, such as anakinra.

In some embodiments, the additional therapeutic agent is a TREM receptor 1 antagonist, such as nangibotide.

In some embodiments, the additional therapeutic agent is a trypsin inhibitor, such as ulinastatin.

In some embodiments, the additional therapeutic agent is a tubulin inhibitor such as sabizabulin, CCI-001, PCNT-13, CR-42-24, albendazole, entasobulin, SAR-132885, or ON-24160.

In some embodiments, the additional therapeutic agent is a VIP receptor agonist, such as aviptadil.

In some embodiments, the additional therapeutic agent is a xanthine oxidase inhibitor, such as oxypurinol.

In some embodiments, the additional therapeutic agent is a vasodilator, such as iloprost, epoprostenol (VentaProst), zavegepant, TXA-127, USB-002, ambrisentan, nitric oxide nasal spray (NORS), pentoxifylline, propranolol, RESP301, sodium nitrite, or dipyridamole.

In some embodiments, the additional therapeutic agent is a vitamin D3 receptor agonist, such as cholecalciferol.

In some embodiments, the additional therapeutic agent is a zonulin inhibitor, such as larazotide acetate.

In some embodiments, the additional therapeutic agent is a synthetic retinoid derivative, such as fenretinide.

In some embodiments, the additional therapeutic agent is a glucose metabolism inhibitor such as WP-1122 or WP-1096.

In some embodiments, the additional therapeutic agent is adalimumab, AT-H201, 2-deoxy-D-glucose, AD-17002, AIC-649, AMTX-100, astodrimer, AZD-1656, belapectin, bitespiramycin, bucillamine, budesonide, CNM-AgZn-17, Codivir, CT-38, danicopan, didodecyl methotrexate, DW-2008S (DW-2008), EDP-1815, EG-009A, Fabencov, Gamunex, genistein, GLS-1200, hzVSF-v13, imidazolyl ethanamide pentandioic acid, IMM-101, MAS-825, MRG-001, Nasitrol, Nylexa, olverembatinib, OP-101, OPN-019, Orynotide rhesus theta defensin-1, pyronaridine+artesunate, dapsone, RPH-104, sodium pyruvate, Sulforadex, tafenoquine, TB-006, telacebec, Tempol, TL-895, thimesoral, trimodulin, XC-221, XC-7, zunsemetinib, metformin glycinate, lucinactant, EOM-613, mosedipimod, ivermectin, leflunomide, ibudilast, RBT-9, raloxifene, prothione, gemcabene, or idronoxil.

In some embodiments, the additional therapeutic agent is a CD73 antagonist, such as AK-119.

In some embodiments, the additional therapeutic agent is a CD95 protein fusion, such as asunercept.

In some embodiments, the additional therapeutic agent is a complement factor C2 modulator, such as ARGX-117.

In some embodiments, the additional therapeutic agent is a complement C3 inhibitor, such as AMY-101 or NGM-621.

In some embodiments, the additional therapeutic agent is a CXC10 chemokine ligand inhibitor, such as EB-06.

In some embodiments, the additional therapeutic agent is a cytotoxic T-lymphocyte protein-4 fusion protein, such as abatacept

In some embodiments, the additional therapeutic agent is an anti-S. Aureus antibody, such as tosatoxumab.

In some embodiments, the additional therapeutic agent is an anti-LPS antibody, such as IMM-124-E.

In some embodiments, the additional therapeutic agent is an adrenomedullin ligand inhibitor, such as enibarcimab.

In some embodiments, the additional therapeutic agent is a basigin inhibitor, such as meplazumab.

In some embodiments, the additional therapeutic agent is a CD3 antagonist, such as foralumab.

In some embodiments, the additional therapeutic agent is a connective tissue growth factor ligand inhibitor, such as PRS-220, pamrevlumab.

In some embodiments, the additional therapeutic agent is a complement C5a factor inhibitor, such as BDB-1 or vilobelimab.

In some embodiments, the additional therapeutic agent is a complement C5 factor inhibitor, such as ravulizumab.

In some embodiments, the additional therapeutic agent is a mannan-binding lectin serine protease-2 inhibitor, such as narsoplimab.

In some embodiments, the additional therapeutic agent is a GM-CSF modulator, such as STSA-1005, gimsilumab, namilumab, plonmarlimab, otilimab, or lenzilumab.

In some embodiments, the additional therapeutic agent is a heat shock protein inhibitor/IL-6 receptor antagonist, such as siltuximab.

In some embodiments, the additional therapeutic agent is an IL-6 receptor antagonist, such as clazakizumab, levilimab, olokizumab, tocilizumab, or sirukumab,

In some embodiments, the additional therapeutic agent is an IL-8 receptor antagonist, such as BMS-986253.

In some embodiments, the additional therapeutic agent is an interleukin-1 beta ligand inhibitor, such as canakinumab.

In some embodiments, the additional therapeutic agent is an interferon gamma ligand inhibitor, such as emapalumab.

In some embodiments, the additional therapeutic agent is an anti-ILT7 antibody, such as daxdilimab.

In some embodiments, the additional therapeutic agent is a monocyte differentiation antigen CD14 inhibitor, such as atibuclimab.

In some embodiments, the additional therapeutic agent is a plasma kallikrein inhibitor, such as lanadelumab.

In some embodiments, the additional therapeutic agent is a platelet glycoprotein VI inhibitor, such as glenzocimab.

In some embodiments, the additional therapeutic agent is a T-cell differentiation antigen CD6 inhibitor, such as itolizumab.

In some embodiments, the additional therapeutic agent is a TNF alpha ligand inhibitor/TNF binding agent, such as infliximab.

In some embodiments, the additional therapeutic agent is an anti-LIGHT antibody, such as AVTX-002.

In some embodiments, the additional therapeutic agent is IMC-2 (valacyclovir+celecoxib), or AXA-1125.

In some embodiments, the additional therapeutic agent is COVID-HIG.

In some embodiments, a solid form of the disclosure, or a pharmaceutically acceptable salt thereof, is co-administered with one or more agents useful for the treatment and/or prophylaxis of COVID-19.

Non-limiting examples of such agents include corticosteroids, such as dexamethasone, hydrocortisone, methylprednisolone, or prednisone; interleukin-6 (IL-6) receptor blockers, such as tocilizumab or sarilumab; Janus kinase (JAK) inhibitors, such as baricitinib, ruxolitinib, or tofacitinib; and antiviral agents, such as molnupiravir, sotrovimab, or remdesivir.

In further embodiments, a solid form of the disclosure, or a pharmaceutically acceptable salt thereof, is co-administered with two or more agents useful for the treatment of COVID-19. Agents useful for the treatment and/or prophylaxis of COVID-19 include but are not limited to a solid form of the disclosure and two additional therapeutic agents, such as nirmatrelvir and ritonavir, casirivimab and imdevimab, or ruxolitinib and tofacitinib.

In some embodiments, the additional therapeutic agent is an antiviral agent. In some embodiments, the antiviral agent is an entry inhibitor. In some embodiments, the antiviral agent is a protease inhibitor. In some embodiments, the antiviral agent is an RNA polymerase inhibitor. In some embodiments, the additional therapeutic agent is an RNA-dependent RNA polymerase (RdRp) inhibitor.

In some embodiments, the antiviral agent is selected from angiotensin converting enzyme 2 inhibitors, angiotensin converting enzyme 2 modulators, angiotensin converting enzyme 2 stimulators, angiotensin II AT-2 receptor agonists, angiotensin II AT-2 receptor antagonists, angiotensin II receptor modulators, coronavirus nucleoprotein modulators, coronavirus small envelope protein modulators, coronavirus spike glycoprotein inhibitors, coronavirus spike glycoprotein modulators, SARS-COV-2 envelope small membrane protein inhibitors, SARS-COV-2 envelope small membrane protein modulators, SARS-COV-2 MPro inhibitors, SARS-COV-2 nonstructural protein 8 modulators, SARS-COV-2 nucleoprotein inhibitors, SARS-CoV-2 nucleoprotein modulators, SARS-COV-2 protein 3a inhibitors, SARS-CoV-2 replicase polyprotein 1a inhibitors, SARS-COV-2 replicase polyprotein 1a modulators, SARS-COV-2 replicase polyprotein lab inhibitors, SARS-COV-2 replicase polyprotein lab modulators, SARS-COV-2 spike glycoprotein inhibitors, SARS-COV-2 spike glycoprotein modulators, SARS-COV-2 structural glycoprotein modulators, papain inhibitors, protease inhibitors, protease modulators, RNA polymerase inhibitors, RNA polymerase modulators, RNA-dependent RNA polymerase (RdRp) inhibitors, SARS coronavirus 3C protease like inhibitors, SARS-COV-2 nsp14 methyltransferase enzyme inhibitor, 3CLpro/Mpro inhibitors, serine protease inhibitors, transmembrane serine protease 2 inhibitors, transmembrane serine protease 2 modulators, viral envelope protein inhibitors, viral protease inhibitors, viral protease modulators, viral protein target modulators, viral ribonuclease inhibitors, and viral structural protein modulators.

In some embodiments, the additional therapeutic agent is an entry inhibitor. For example, in some embodiments the additional therapeutic agent is an ACE2 inhibitor, a fusion inhibitor, or a protease inhibitor.

In some embodiments, the additional therapeutic agent is an angiotensin converting enzyme 2 inhibitor, such as SBK-001.

In some embodiments, the additional therapeutic agent is an angiotensin converting enzyme 2 modulator, such as neumifil or JN-2019.

In some embodiments, the additional therapeutic agent is an entry inhibitor such as MU-UNMC-1, or SAI-4.

In some embodiments, the additional therapeutic agent is an angiotensin converting enzyme 2 stimulator, such as alunacedase alfa.

In some embodiments, the additional therapeutic agent is an angiotensin II AT-2 receptor agonist, such as VP-01.

In some embodiments, the additional therapeutic agent is an ACE II receptor antagonist, such as DX-600.

In some embodiments, the additional therapeutic agent is an angiotensin II receptor modulator, such as TXA-127.

In some embodiments, the additional therapeutic agent is a transmembrane serine protease 2 modulator, such as BC-201, N-0385.

In some embodiments, the additional therapeutic agent is a viral envelope protein inhibitor, such as MXB-9 or MXB-004.

In some embodiments, the additional therapeutic agent is a RNAi agent such as ARO-COV or SNS-812.

In some embodiments, the additional therapeutic agent is a vaccine. For example, in some embodiments, the additional therapeutic agent is a DNA vaccine, RNA vaccine, live-attenuated vaccine, inactivated vaccine (i.e., inactivated SARS-COV-2 vaccine), therapeutic vaccine, prophylactic vaccine, protein-based vaccine, viral vector vaccine, cellular vaccine, or dendritic cell vaccine.

In some embodiments, the additional therapeutic agent is a vaccine such as tozinameran, NVX-CoV2373, elasomeran, KD-414, Ad26.COV2-S, Vaxzevria, SCB-2019, AKS-452, VLA-2001, HDT-301, S-268019, MVC-COV1901, mRNA-1273.214, mRNA-1273.213, mRNA-1273.222, NVX-CoV2515, Covaxin, BBIBP-CorV, GBP-510, mRNA-1273.351+mRNA-1273.617 (SARS-COV-2 multivalent mRNA vaccine, COVID-19), Ad5-nCoV, Omicron-based COVID-19 vaccine (mRNA vaccine, COVID-19), mRNA-1073, mRNA-1273.214, mRNA-1230, mRNA-1283, Omicron-based COVID-19 vaccine, SARS-COV-2 Protein Subunit Recombinant Vaccine, Sputnik M, ZyCoV-D, COVID-19 XWG-03, mRNA-1273.529, mRNA-1010, CoronaVac, AZD-2816, Sputnik V, inactivated SARS-COV-2 vaccine (Vero cell, COVID-19), DS-5670, PHH-IV, INO-4800, UB-612, coronavirus vaccine (whole-virion, inactivated/purified), ReCOV, MT-2766, ARCT-154, SP-0253, CORBEVAX, mRNA-1273.211, ZF-2001, Sputnik Light, recombinant protein vaccine (COVID-19/SARS-COV-2 infection), VSV vector-based vaccine targeting spike glycoprotein (COVID-19), VLA-2101, GRT-R912, GRAd-COV2, VPM-1002, COViran Barekat, Ad5-nCoV-IH, ARCoV, Covax-19, recombinant SARS-COV-2 vaccine (protein subunit/CHO cell, COVID-19), BBV-154, RAZI Cov Pars, COVID-19 vaccine (inactivated/Vero cells/intramuscular, SARS-COV-2 infection), COVID-19 vaccine (inactivated, Vero cells/intramuscular), BNT-162b2s01, BNT-162b4, BNT-162b5, BNT-162b2 Omi, BNT-162b2 bivalent, CIGB-66, mRNA-1273.617, Mycobacterium w, ERUCOV-VAC, AG-0301-COVID19, fakhravac, AV-COVID-19, peptide vaccine (COVID-19), Nanocovax, SARS-COV-2 vaccine (inactivated/Vero cells/intramuscular, COVID-19), QAZCOVID-IN, S-875670 nasal vaccine, VTP-500, or BNT162b5.

In some embodiments, the additional therapeutic agent is a protease inhibitor. For example, in some embodiments the additional therapeutic agent is a 3C-like cysteine protease inhibitor (3CLpro, also called Main protease, Mpro), a papain-like protease inhibitor (PLpro), serine protease inhibitor, or transmembrane serine protease 2 inhibitor (TMPRSS2),

In some embodiments, the additional therapeutic agent is a 3CLpro/Mpro inhibitor, such as ABBV-903, AB-343, CDI-873, GC-373, GC-376, pomotrelvir (PBI-0451), UCI-1, bofutrelvir (FB-2001, DC-402234), DC-402267, GDI-4405, HS-10517, RAY-1216, MPI-8, SH-879, SH-580, EDP-235, VV-993, CDI-988, MI-30, nirmatrelvir, ensitrelvir, ASC-11, ASC-11+ritonavir, EDDC-2214, SIM-0417, PF-07817883, simnotrelvir, simnotrelvir+ritonavir, SYH-2055, ISM-3312, CDI-45205, LHP-803 (COR-803), ALG-097111, TJC-642, CVD-0013943, olgotrelvir (STI-1558), eravacycline, cynarine, WPV-01, or prexasertib.

In some embodiments, the additional therapeutic agent is a papain-like protease inhibitor (PLpro), such as SBFM-PL4 or GRL-0617.

In some embodiments, the additional therapeutic agent is a SARS-COV-2 helicase Nsp13 inhibitor, such as EIS-4363.

In some embodiments, the additional therapeutic agent is a SARS-COV-2 helicase Nsp14 inhibitor, such as TO-507.

In some embodiments, the additional therapeutic agent is a SARS-COV-2 spike(S) and protease modulator, such as ENU-200.

In some embodiments, the additional therapeutic agent is a protease inhibitor, such as ALG-097558 or MRX-18.

In some embodiments, the additional therapeutic agent is a serine protease inhibitor, such as upamostat, nafamostat, camostat mesylate, nafamostat mesylate, or camostat.

In some embodiments, the additional therapeutic agent is a 3CLpro/transmembrane serine protease 2 inhibitor, such as SNB-01 (pentarlandir) or SNB-02.

In some embodiments, the additional therapeutic agent is a viral protease inhibitor, such as Pan-Corona, Cov-X, or bepridil.

In some embodiments, the additional therapeutic agent is an RNA polymerase inhibitor. For example, in some embodiments, the additional therapeutic agent is an RNA polymerase inhibitor, or an RNA-dependent RNA polymerase (RdRp) inhibitor.

In some embodiments, the additional therapeutic agent is an RNA-dependent RNA polymerase (RdRp) inhibitor, such as remdesivir, NV-CoV-2, NV-CoV-2-R, NV-CoV-1 encapsulated remdesivir, CMX-8521, GS-621763, GS-5245, GS-441524, DEP remdesivir, ATV-006, deuremidevir (VV-116), LGN-20, CMX-521, SHEN-26, MB-905, and compounds disclosed in WO2022142477, WO2021213288, WO2022047065.

In some embodiments, the additional therapeutic agent is an RNA polymerase inhibitor, such as molnupiravir (EIDD-2801), favipiravir, bemnifosbuvir, sofosbuvir, ASC-10, or galidesivir.

In some embodiments, the additional therapeutic agent is viral entry inhibitor, such as brilacidin.

In some embodiments, the additional therapeutic agent is an antibody that binds to a coronavirus, for example an antibody that binds to SARS or MERS.

In some embodiments, the additional therapeutic agent is an antibody, for example a monoclonal antibody. For example, the additional therapeutic agent is an antibody against SARS-COV-2, neutralizing nanobodies, antibodies that target the SARS-COV-2 spike protein, fusion proteins, multispecific antibodies, and antibodies that can neutralize SARS-COV-2 (SARS-COV-2 neutralizing antibodies).

In some embodiments, the additional therapeutic agent is an antibody that targets specific sites on ACE2. In some embodiments, the additional therapeutic agent is a polypeptide targeting SARS-COV-2 spike protein (S-protein).

In some embodiments, the additional therapeutic agent is a SARS-COV-2 virus antibody.

In some embodiments, the antibody is ABBV-47D11, COVI-GUARD (STI-1499), C144-LS+C135-LS, DIOS-202, DIOS-203, DIOS-301, DXP-604, JMB-2002, LY-CovMab, bamlanivimab (LY-CoV555), GIGA-2050, IBI-314, S309, SAB-185, etesevimab (CB6), COR-101, JS016, VNAR, VIR-7832 and/or sotrovimab (VIR-7831), casirivimab+imdevimab (REGN-COV2 or REGN10933+RGN10987), BAT2020, BAT2019, 47D11, YBSW-015, or PA-001.

In some embodiments, the additional therapeutic agent is STI-9199 (COVI-SHIELD), STI-9167 or AR-701 (AR-703 and AR-720).

In some embodiments, the additional therapeutic agent is BRII-196, BRII-198, ADG-10, adintrevimab (ADG-20), ABP-300, BA-7208, BI-767551, BHV-1200, CT-P63, JS-026, sotrovimab (GSK-4182136), tixagevimab+cilgavimab (AZD-7442), regdanvimab, SAB-301, AOD-01, plutavimab (COVI-AMG), 9MW-3311 (MW-33), DXP-593, BSVEQAb, anti-SARS-CoV-2 IgY, COVID-EIG, CSL-760, F-61, REGN-3048-3051, SARS-COV-2 monoclonal antibodies (COVID-19, ADM-03820), enuzovimab (HFB-30132A), INM-005, SCTA01, TY-027, XAV-19, amubarvimab+romlusevimab, SCTA-01, bebtelovimab, beludavimab, IBI-O123, IGM-6268. FYB-207, FS-2101, RBT-0813, REGN-14256, REGN-14284, SPKM-001, XVR-011, TB202-3, TB181-36, TB339-031, LMN-301, LQ-050, COVAB-36, MAD-0004J08, STI-2099, TATX-03, TZLS-501, ZCB-11, AZD-3152, VYD-222, XVR-012, or ACV-200-17.

In some embodiments, the additional therapeutic agent is an engineered ACE-2-IgG1-Fc-fusion protein targeting SARS-Cov-2 RBD, such as EU-129, bivalent ACE2-IgG Fc null fusion protein (SI-F019).

In some embodiments, the additional therapeutic agent is an ACE2-Fc receptor fusion protein, such as HLX-71.

In some embodiments, the additional therapeutic agent is ensovibep.

In some embodiments, the additional therapeutic agent is SYZJ-001.

In some embodiments, the additional therapeutic agent is an HIV-1 protease inhibitor, such as ASC-09F (ASC-09+ritonavir) or lopinavir+ritonavir.

In some embodiments, the additional therapeutic agent is a non-nucleoside reverse transcriptase inhibitor, such as elsulfavirine.

In some embodiments, the additional therapeutic agent is a nucleoside reverse transcriptase inhibitor, such as azvudine.

In some embodiments, the additional therapeutic agent is Abbv-990, ABBV-903, 2b-11, 5-aminolevulinic phosphoric acid, AGP-600, AGM-380, AIP-502, ALG-150150, BAT-2022, NED-260, burfiralimab, ALG-097431, bardoxolone, BW-PS-119, clofoctol, CR-405, delcetravir, D4-102-01, D4-102-02, ESFAM-289, ENOB-CV-01, ENOB-CV-11, EIS-10700, EV-300, beta-521, GEA-001, SIM-0417, molnupiravir, Pan-Corona, Tollovir, nirmatrelvir+ritonavir (Paxlovid®), JTBC-00201, favipiravir, favipiravir+cathepsin inhibitor (TNX-3900), GC-376, upamostat, LeSoleil-01, LeSoleil-02+, benfovir, VV-116, VV-993, SNB-01, EDP-235, Cov-X, ensitrelvir, MPI-8, masitinib, ALG-097558, ASC-11, PBI-0451, nafamostat, nafamostat mesylate, CDI-45205, LHP-803 (COR-803), ALG-097111, BC-201, SH-879, CDI-873, CDI-988, remdesivir, NV-CoV-2-R, NV-CoV-1 encapsulated remdesivir, NA-831+remdesivir, DEP remdesivir, GS-621763, GS-5245, GLS-5310, bemnifosbuvir, QLS-1128, ASC-10, SBFM-PL4, camostat mesylate, UCI-1, FB-2001 (DC-402234), ebselen, SH-580, LeSoleil-01, LeSoleil-02+, MRX-18, MXB-9, MI-09, MI-30, SNB-02, SJP-002C, TJC-642, ENU-200, CVD-0013943, GS-441524, bepridil, MXB-004, eravacycline, GRL-0617, GST-HG171, GST-HG171+ritonavir, camostat, GC-373, KD-1, nitazoxanide, cynarine, prexasertib, PL-M, RAY-1216, SACT-COVID-19, MP-18, EIDD-1931, EDDC-2214, nitric oxide, apabetalone, AnQlar, SBK-001, LQ-050, CG-SpikeDown, bamlanivimab, JTBC-00101, HLX-71, HT-002, HY-209, HY-3000, HSC-1553, FYB-207, ensovibep, SYZJ-001, EU-129, neumifil, JN-2019 (KG-2019), LCB-99, AR-701, vostesyl, PLM-402, PJS-539, CTB-ACE2, TB181-36, TB202-3, ABP-300, XVR-011, MSP-008-22, MU-UNMC-1, MU-UNMC-2, MIC-1930, MLT-103, Mpro inhibitors (Anixa Biosciences), PBF-4554, alunacedase alfa, VP-01, TRV-027, DX-600,TXA-127, NVX-CoV2515, raphamin, RCYM-002, RCYM-003, riamilovir, SARS-Cov-2 PL pro inhibitor (Enanta), SBP-502, SM-4, STB-R040, THY-01, tozinameran, elasomeran, Ad5-nCOV, BBIBP-CorV, CoronaVac, MVC-COV1901, NVX-CoV2373, sotrovimab, Sputnik V, TEE-001, Tyme-19, Vaxzevria, XW-001, ZF-2001, or ZyCoV-D.

In some embodiments, the additional therapeutic or prophylactic agent is a SARS-CoV-2 MPro inhibitor. In some embodiments, the SARS-COV-2 MPro inhibitor is nirmatrelvir. In some embodiments, the SARS-COV-2 MPro inhibitor is ritonavir.

It is also possible to combine any compound of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds described herein before or after administration of unit dosages of one or more other active therapeutic agents, for example, administration of the compounds described herein within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect, which is greater than the predicted purely additive effects of the individual compounds of the combination.

The compounds described herein are also used in combination with other active therapeutic agents. For the treatment of Pneumoviridae virus infections, preferably, the other active therapeutic agent is active against Pneumoviridae virus infections, particularly respiratory syncytial virus infections and/or metapneumovirus infections. Non-limiting examples of these other active therapeutic agents active against RSV are ribavirin, palivizumab, motavizumab, RSV-IGIV (RespiGam®), MEDI-557, A-60444 (also known as RSV604), MDT-637, BMS-433771, ALN-RSVO, ALX-0171 and mixtures thereof. Other non-limiting examples of other active therapeutic agents active against respiratory syncytial virus infections include respiratory syncytial virus protein F inhibitors, such as AK-0529; RV-521, ALX-0171, JNJ-53718678, BTA-585, and presatovir; RNA polymerase inhibitors, such as lumicitabine and ALS-8112; anti-RSV G protein antibodies, such as anti-G-protein mAb; viral replication inhibitors, such as nitazoxanide.

In some embodiments, the other active therapeutic agent may be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGP084-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent may be a vaccine for the treatment or prevention of metapneumovirus infections, including but not limited to mRNA-1653 and rHMPV-Pa vaccine.

The compounds described herein are also used in combination with other active therapeutic agents. For the treatment of picornaviridae virus infections, preferably, the other active therapeutic agent is active against picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody.

In some embodiments, the other active therapeutic agent may be a vaccine for the treatment or prevention of picornaviridae virus infections, including but not limited to EV71 vaccines, TAK-021, and EV-D68 adenovector-based vaccine.

Many of the infections of the pneumoviridae, picornaviridae, and coronaviridae viruses are respiratory infections. Therefore, additional active therapeutics used to treat respiratory symptoms and sequelae of infection may be used in combination with the compounds described herein. The additional agents are preferably administered orally or by direct inhalation. For example, other preferred additional therapeutic agents in combination with the compounds described herein for the treatment of viral respiratory infections include, but are not limited to, bronchodilators and corticosteroids.

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the compounds described herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

Other anti-inflammatory agents working through anti-inflammatory cascade mechanisms are also useful as additional therapeutic agents in combination with the compounds described herein for the treatment of viral respiratory infections. Applying “anti-inflammatory signal transduction modulators” (referred to in this text as AIS™), like phosphodiesterase inhibitors (e.g., PDE-4, PDE-5, or PDE-7 specific), transcription factor inhibitors (e.g., blocking NFκB through IKK inhibition), or kinase inhibitors (e.g., blocking P38 MAP, JNK, PI3K, EGFR or Syk) is a logical approach to switching off inflammation as these small molecules target a limited number of common intracellular pathways-those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-y]-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-y]-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

Combinations comprising inhaled B2-adrenoreceptor agonist bronchodilators such as formoterol, albuterol or salmeterol with the compounds described herein are also suitable, but non-limiting, combinations useful for the treatment of respiratory viral infections.

Combinations of inhaled B2-adrenoreceptor agonist bronchodilators such as formoterol or salmeterol with ICS's are also used to treat both the bronchoconstriction and the inflammation (Symbicort® and Advair®, respectively). The combinations comprising these ICS and B2-adrenoreceptor agonist combinations along with the compounds described herein are also suitable, but non-limiting, combinations useful for the treatment of respiratory viral infections.

Other examples of Beta 2 adrenoceptor agonists are bedoradrine, vilanterol, indacaterol, olodaterol, tulobuterol, formoterol, abediterol, salbutamol, arformoterol, levalbuterol, fenoterol, and TD-5471.

For the treatment or prophylaxis of pulmonary broncho-constriction, anticholinergics are of potential use and, therefore, useful as an additional therapeutic agent in combination with the compounds described herein for the treatment of viral respiratory infections. These anticholinergics include, but are not limited to, antagonists of the muscarinic receptor (particularly of the M3 subtype) which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1] octane (Ipratropium-N,N-diethylglycinate); 1-Cyclohexyl-3,4-dihydro-IH-isoquinoline-2-carboxylic acid 1-aza-bicyclo[2.2.2] oct-3-yl ester (Solifenacin); 2-Hydroxymethyl-4-methanesulfinyl-2-phenyl-butyric acid 1-aza-bicyclo[2.2.2] oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4] nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4] nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2] oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, bencycloquidium bromide.

The solid forms described herein may also be combined with mucolytic agents to treat both the infection and symptoms of respiratory infections. A non-limiting example of a mucolytic agent is ambroxol. Similarly, the compounds may be combined with expectorants to treat both the infection and symptoms of respiratory infections. A non-limiting example of an expectorant is guaifenesin.

Nebulized hypertonic saline is used to improve immediate and long-term clearance of small airways in patients with lung diseases (Kuzik, J. Pediatrics 2007, 266). Thus, the compounds described herein may also be combined with nebulized hypertonic saline particularly when the virus infection is complicated with bronchiolitis. The combination of the compound described herein with hypertonic saline may also comprise any of the additional agents discussed above. In one embodiment, nebulized about 3% hypertonic saline is used.

The solid forms provided herein are also used in combination with other active therapeutic agents. For the treatment of flaviviridae virus infections, preferably, the other active therapeutic agent is active against flaviviridae virus infections.

For treatment of the dengue virus infection, non-limiting examples of the other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent may be a vaccine for the treatment or prevention of dengue, including but not limited to TetraVax-DV, Dengvaxia®, DPIV-001, TAK-003, live attenuated dengue vaccine, tetravalent dengue fever vaccine, tetravalent DNA vaccine, rDEN2delta30-7169; and DENV-1 PIV.

The solid forms described herein are also used in combination with other active therapeutic agents. For the treatment of filoviridae virus infections, preferably, the other active therapeutic agent is active against filoviridae virus infections, particularly Marburg virus, Ebola virus and Cueva virus infections. Non-limiting examples of these other active therapeutic agents are: ribavirin, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl) pyrrolidine-3,4-diol), TKM-Ebola, T-705 monophosphate, T-705 diphosphate, T-705 triphosphate, FGI-106 (1-N,7-N-bis [3-(dimethylamino) propyl]-3,9-dimethylquinolino [8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as REGN-3470-3471-3479 and ZMapp.

Other non-limiting active therapeutic agents active against Ebola include an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPCI inhibitors).

In some embodiments, the other active therapeutic agent may be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The solid forms described herein may also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The solid forms described herein are also intended for use with general care provided to patients with filoviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

The invention will be described in greater detail by way of specific examples. The following examples are offered for illustrative purposes and are not intended to limit the invention in any manner. Those of skill in the art will readily recognize a variety of non-critical parameters, which can be changed or modified to yield essentially the same results.

### Examples

### Abbreviations

XRPD X-ray Powder Diffraction
DSC Differential Scanning calorimetry
TGA Thermogravimetric Analysis
DVS Dynamic Vapor Sorption

### General

XRPD patterns were collected with a PANalytical X′Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X′Celerator) located 240 mm from the sample and Data Collector software v. 2.2b.

Differential Scanning calorimetry (DSC) data were collected using a TA Instruments Q2000 differential scanning calorimeter. Temperature calibration was performed using NIST-traceable indium metal. The sample was placed into a T zero aluminum DSC pan, covered with a lid pierced using a needle. The weight was then accurately recorded. A weighed aluminum pan configured as the sample pan was placed on the reference side of the cell. The sample was heated from 20° C. to 300° C. at 10° C./minute.

Thermogravimetric Analysis (TGA) data were collected using a TA Instruments Q5000 thermogravimetric analyzer. Temperature calibration was performed using nickel and Alumel™. Each sample was placed in an aluminum pan and inserted into the TG furnace. The furnace was heated under a nitrogen purge. The sample was heated from ambient to 300° C. at 10° C./minute.

### Example 1. Formula I Maleate

### Preparation

Formula I maleate was first prepared by suspending about 40 mg of Formula I freebase Form III (as described in WO2022047065) in 0.4 mL of acetonitrile. Approximately one molar equivalent of maleic acid was added to the suspension and the resulting slurry was stirred at ambient temperature for about one day. The solids were isolated by centrifugation and dried in the vacuum oven at about 40° C.

The XRPD pattern of Formula I maleate is shown in FIG. 1 it is characterized by Tier 1 reflections at 3.9°, 7.7°, and 19.4° 2θ, but also Tier 2 at 2.2°, 15.2°, and 27.0° 2θ, and Tier 3 at 11.6°, 12.2°, and 28.1° 2θ.

The DSC thermogram is shown in FIG. 2 and displays two endothermic transitions at about 155° C. and 161° C. The TGA thermogram is shown in FIG. 3 and indicates that the phase loses about 0.5% of its weight before degrading.

TABLE 1
XRPD peak list for Formula I maleate by tiers

| Tier 1 | | Tier 2 | | Tier 3 | |
|---|---|---|---|---|---|
| Position | Relative | Position | Relative | Position | Relative |
| ° 2θ | intensity (%) | ° 2θ | intensity (%) | ° 2θ | intensity (%) |
| 3.9 | 100 | 2.2 | 3 | 11.6 | 1 |
| 7.7 | 10 | 15.2 | 2 | 12.2 | 1 |
| 19.4 | 4 | 27.0 | 2 | 28.1 | 2 |

TABLE 2
Complete XRPD peak list for Formula I maleate

| Pos. [°2Th.] | Rel. Int. [%] |
|---|---|
| 2.2 | 3 |
| 3.9 | 100 |
| 7.7 | 10 |
| 11.6 | 1 |
| 12.2 | 1 |
| 15.2 | 2 |
| 15.8 | 2 |
| 16.8 | 2 |
| 17.9 | 1 |
| 18.7 | 1 |
| 19.4 | 4 |
| 22.9 | 1 |
| 25.2 | 1 |
| 26.0 | 1 |
| 27.0 | 2 |
| 28.1 | 2 |
| 31.8 | 1 |

### Example 2. Formula I Oxalate

### Preparation

Formula I oxalate was prepared by suspending about 40 mg of Formula I freebase Form III (as described in WO2022047065) in 0.4 mL of acetonitrile. Approximately two molar equivalent of oxalic acid was added to the suspension and the resulting slurry was stirred at ambient temperature for about one day. The solids were isolated by centrifugation and dried in the vacuum oven at about 40° C.

### Characterization

The XRPD pattern of Formula I oxalate is presented in FIG. 4. It is characterized by Tier 1 reflections at 3.5°, 7.0°, and 17.7° 2θ, but also Tier 2 at 10.2°, 19.5°, and 27.0° 2θ, and Tier 3 at 12.9°, 21.2°, and 29.5° 2θ.

The DSC thermogram of Formula I oxalate is shown in FIG. 5. It shows two endothermic events around 92° C. and 128° C. These events are preceded by a broad endothermic event which is likely due to solvent loss. The TGA thermogram is presented in FIG. 6. It shows that the material lose about 2.7% of its weight before degrading.

TABLE 3
XRPD peak list for Formula I oxalate by tiers

| Tier 1 | | Tier 2 | | Tier 3 | |
|---|---|---|---|---|---|
| Position | Relative | Position | Relative | Position | Relative |
| ° 2θ | intensity (%) | ° 2θ | intensity (%) | ° 2θ | intensity (%) |
| 3.5 | 100 | 10.2 | 3 | 12.9 | 1 |
| 7.0 | 12 | 19.5 | 2 | 21.2 | 1 |
| 17.7 | 2 | 27.0 | 2 | 29.5 | 1 |

TABLE 4
Complete XRPD peak list for Formula I oxalate

| Pos. [°2Th.] | Rel. Int. [%] |
|---|---|
| 3.5 | 100 |
| 7.0 | 12 |
| 10.2 | 3 |
| 12.9 | 1 |
| 16.8 | 1 |
| 17.7 | 2 |
| 19.5 | 2 |
| 21.2 | 1 |
| 26.5 | 1 |
| 27.0 | 2 |
| 27.7 | 1 |
| 29.5 | 1 |
| 32.2 | 1 |

### Example 3. Formula I Methanesulfonate Material A

### Preparation

Formula I methanesulfonate Material A was prepared by suspending about 40 mg of Formula I freebase Form III (as described in WO2022047065) in 0.4 mL of acetonitrile. Approximately one molar equivalent of methanesulfonic acid to the suspension and the resulting mixture was stirred at ambient temperature for about one day. No slurry was obtained. The acetonitrile was removed by rotary evaporation and 0.4 mL of ethanol was added. No slurry was obtained. The ethanol was removed by rotary evaporation and 0.4 mL of tetrahydrofuran (THF) was added. After about a day a slurry was obtained. The solids were isolated by centrifugation and dried in the vacuum oven at about 40° C.

The XRPD pattern of Formula I methanesulfonate Material A is shown in FIG. 7. It is characterized by Tier 1 reflections at 8.1°, 14.7°, and 21.4° 2θ, but also Tier 2 at 16.3°, 19.9°, and 24.7° 2θ, and Tier 3 at 19.0°, 24.2°, and 27.9° 2θ.

TABLE 5
XRPD peak list for Formula I methanesulfonate Material A by tiers

| Tier 1 | | Tier 2 | | Tier 3 | |
|---|---|---|---|---|---|
| Position | Relative | Position | Relative | Position | Relative |
| ° 2θ | intensity (%) | ° 2θ | intensity (%) | ° 2θ | intensity (%) |
| 8.1 | 69 | 16.3 | 35 | 19.0 | 20 |
| 14.7 | 100 | 19.9 | 26 | 24.2 | 31 |
| 21.4 | 59 | 24.7 | 39 | 27.9 | 37 |

TABLE 6
Complete XRPD peak list for Formula
I methanesulfonate Material A

| Pos. [°2Th.] | Rel. Int. [%] |
|---|---|
| 8.1 | 69 |
| 13.1 | 3 |
| 14.7 | 100 |
| 16.3 | 35 |
| 17.0 | 12 |
| 18.4 | 14 |
| 18.7 | 11 |
| 19.0 | 20 |
| 19.9 | 26 |
| 21.4 | 59 |
| 22.2 | 21 |
| 23.0 | 9 |
| 24.2 | 31 |
| 24.7 | 39 |
| 27.1 | 19 |
| 27.6 | 32 |
| 27.9 | 37 |
| 28.7 | 21 |
| 29.4 | 7 |
| 33.1 | 14 |
| 33.5 | 15 |
| 33.9 | 11 |
| 34.3 | 10 |
| 34.9 | 8 |
| 37.2 | 5 |
| 38.5 | 5 |
| 39.0 | 6 |
| 39.3 | 6 |

### Example 4. Formula I Methanesulfonate Material B

### Preparation

Formula I methanesulfonate Material B was prepared by suspending about 200 mg of GS-5245 freebase Form III (as described in WO2022047065) in 2 mL of tetrahydrofuran. Approximately 1.1 molar equivalents of methanesulfonic acid was added. A small amount of Formula I methanesulfonate Material A (Example 3) was added as seeds and the mixture was stirred at about 22° C. After about 2 days, the solvent was removed by rotary evaporation and 0.5 mL of tetrahydrofuran was added and the mixture was stirred at ambient for 2 days. A thick immobile slurry was obtained. The slurry was diluted with 1.75 mL of tetrahydrofuran and isolated by centrifugation. The resulting wet solids were dried at about 40° C. in the vacuum oven.

The XRPD pattern of Formula I methanesulfonate Material B is shown in FIG. 8 and is characterized by Tier 1 reflections at 5.7°, 11.4°, and 18.8° 2θ, but also Tier 2 at 7.9°, 17.1°, and 27.7° 2θ, and Tier 3 at 9.4°, 21.3°, and 24.1° 2θ.

The TGA thermogram is shown in FIG. 9. It shows that the material loses about 1.7% of its weight before degrading.

TABLE 7
XRPD peak list for GS-5245 methanesulfonate Material B by tiers

| Tier 1 | | Tier 2 | | Tier 3 | |
|---|---|---|---|---|---|
| Position | Relative | Position | Relative | Position | Relative |
| ° 2θ | intensity (%) | ° 2θ | intensity (%) | ° 2θ | intensity (%) |
| 5.7 | 77 | 7.9 | 75 | 9.4 | 55 |
| 11.4 | 99 | 17.1 | 70 | 21.3 | 29 |
| 18.8 | 100 | 27.7 | 39 | 24.1 | 27 |

TABLE 8
Complete XRPD peak list for GS-
5245 methanesulfonate Material B

| Pos. [°2Th.] | Rel. Int. [%] |
|---|---|
| 5.7 | 77 |
| 7.9 | 75 |
| 9.4 | 55 |
| 11.4 | 99 |
| 13.6 | 16 |
| 15.2 | 17 |
| 16.0 | 13 |
| 17.1 | 70 |
| 18.8 | 100 |
| 19.5 | 15 |
| 21.3 | 29 |
| 22.0 | 22 |
| 22.6 | 15 |
| 23.2 | 10 |
| 24.1 | 27 |
| 27.7 | 29 |
| 30.2 | 20 |
| 32.3 | 10 |
| 33.4 | 15 |
| 34.2 | 10 |
| 34.7 | 10 |
| 38.4 | 7 |

### Example 5. Formula I Ethanesulfonate

### Preparation

Formula I ethanesulfonate was prepared by suspending about 40 mg of Formula I freebase Form III (as described in WO2022047065) in 0.4 mL of acetonitrile. Approximately one molar equivalent of ethanesulfonic acid to the suspension and the resulting mixture was stirred at ambient temperature for about one day. No slurry was obtained. The acetonitrile was removed by rotary evaporation and 0.4 mL of ethanol was added. No slurry was obtained. The ethanol was removed by rotary evaporation and 0.4 mL of tetrahydrofuran (THF) was added. After about 8 days a slurry was obtained. The solids were isolated by centrifugation and air dried in a fume hood at about 22° C.

### Characterization

The XRPD pattern of Formula I ethanesulfonate is shown in FIG. 10. It is characterized by Tier 1 reflections at 8.0°, 16.1°, and 32.5° 2θ, but also Tier 2 at 15.2°, 21.5°, and 27.6° 2θ, and Tier 3 at 18.6°, 26.1°, and 33.0° 2θ.

TABLE 9
XRPD peak list for GS-5245 HCl ethanesulfonate by tiers

| Tier 1 | | Tier 2 | | Tier 3 | |
|---|---|---|---|---|---|
| Position | Relative | Position | Relative | Position | Relative |
| ° 2θ | intensity (%) | ° 2θ | intensity (%) | ° 2θ | intensity (%) |
| 8.0 | 100 | 15.2 | 7 | 18.6 | 6 |
| 16.1 | 41 | 21.5 | 29 | 26.1 | 7 |
| 32.5 | 31 | 27.6 | 15 | 33.0 | 4 |

TABLE 10
Complete XRPD peak list for GS-5245 ethanesulfonate

| Pos. [°2Th.] | Rel. Int. [%] |
|---|---|
| 8.0 | 100 |
| 15.2 | 7 |
| 16.1 | 41 |
| 17.0 | 3 |
| 18.6 | 6 |
| 20.4 | 2 |
| 21.5 | 29 |
| 24.2 | 60 |
| 26.1 | 7 |
| 27.6 | 15 |
| 28.4 | 4 |
| 28.9 | 3 |
| 32.5 | 31 |
| 33.0 | 4 |
| 34.7 | 2 |
| 35.9 | 4 |
| 38.1 | 3 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A salt of a compound of Formula I: 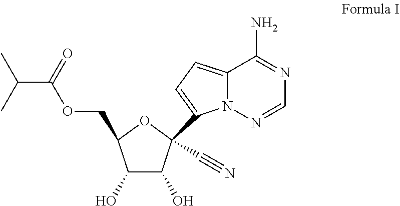 [SMILES: CC(C)C(=O)OC[C@H]1O[C@@](C#N)(c2ccc3c(N)ncnn23)[C@H](O)[C@@H]1O]
wherein the salt is a maleate salt, an oxalate salt, a methanesulfonate salt, or an ethanesulfonate salt.

2. The salt of claim 1, wherein the salt is the maleate salt.

3. A crystalline form of the maleate salt of claim 2, wherein the crystalline form is characterized by an XRPD pattern comprising 2θ-reflections (±0.2 degrees 2θ) at 3.9°, 7.7°, and 19.4°.

4. (canceled)

5. The crystalline form of claim 3, wherein the XRPD pattern comprises 2θ-reflections (±0.2 degrees 2θ) at 2.2°, 3.9°, 7.7°, 15.2°, 19.4° and 27.0°.

6. (canceled)

7. The crystalline form of claim 3, wherein the XRPD pattern comprises 2θ-reflections (±0.2 degrees 2θ) at 2.2°, 3.9°, 7.7°, 11.6°, 12.2°, 15.2°, 19.4°, 27.0°, and 28.1°.

8. The crystalline form of claim 3, wherein the XRPD pattern is substantially as shown in FIG. 1.

9. The crystalline form of claim 3, wherein the crystalline form is characterized by a differential scanning calorimetry pattern comprising a first endothermic transition at about 155° C.

10.-12. (canceled)

13. The salt of claim 1, wherein the salt is the oxalate salt.

14. (canceled)

15. A crystalline form of the oxalate salt of claim 13, wherein the crystalline form is characterized by an XRPD pattern comprising 2θ-reflections (±0.2 degrees 2θ) at 3.5°, 7.0°, and 17.7°.

16. (canceled)

17. The crystalline form of claim 15, wherein the XRPD pattern comprises 2θ-reflections (±0.2 degrees 2θ) at 3.5°, 7.0°, 10.2°, 17.7°, 19.5°, and 27.0°.

18. (canceled)

19. The crystalline form of claim 15, wherein the XRPD pattern comprises degree 2θ-reflections (±0.2 degrees 2θ) at 3.5°, 7.0°, 10.2°, 12.9°, 17.7°, 19.5°, 21.2°, 27.0°, and 29.5°.

20. The crystalline form of claim 15, wherein the XRPD pattern is substantially as shown in FIG. 4.

21. The crystalline form of claim 15, wherein the crystalline form is characterized by a differential scanning calorimetry pattern comprising a first endothermic transition at about 92° C.

22.-24. (canceled)

25. The salt of claim 1, wherein the salt is the methanesulfonate salt.

26. (canceled)

27. A crystalline form of the methanesulfonate salt of claim 25, wherein the crystalline form is characterized by an XRPD pattern comprising 2θ-reflections (±0.2 degrees 2θ) at 8.1°, 14.7°, and 21.4°.

28. (canceled)

29. The crystalline form of claim 27, wherein the XRPD pattern comprises 2θ-reflections (±0.2 degrees 2θ) at 8.1°, 14.7°, 16.3°, 19.9°, 21.4°, and 24.7°.

30. (canceled)

31. The crystalline form of claim 27, wherein the XRPD pattern comprises degree 2θ-reflections (±0.2 degrees 2θ) at 8.1°, 14.7°, 16.3°, 19.0°, 19.9°, 21.4°, 24.2°, 24.7°, and 27.9°.

32. The crystalline form of claim 27, wherein the XRPD pattern is substantially as shown in FIG. 7.

33. A crystalline form of the methanesulfonate salt of claim 25, wherein the crystalline form is characterized by an XRPD pattern comprising 2θ-reflections (±0.2 degrees 2θ) at 5.7°, 11.4°, and 18.8°.

34. (canceled)

35. The crystalline form of claim 33, wherein the XRPD pattern comprises 2θ-reflections (±0.2 degrees 2θ) at 5.7°, 7.9°, 11.4°, 17.1°, 18.8°, and 27.7°.

36. (canceled)

37. The crystalline form of claim 33, wherein the XRPD pattern comprises degree 2θ-reflections (±0.2 degrees 2θ) at 5.7°, 7.9°,9.4°, 11.4°, 17.1°, 18.8°, 21.3°, 24.1°, and 27.7°.

38. The crystalline form of claim 33, wherein the XRPD pattern is substantially as shown in FIG. 8.

39. (canceled)

40. The salt of claim 1, wherein the salt is the ethanesulfonate salt.

41. (canceled)

42. A crystalline form of the ethanesulfonate salt of claim 40, wherein the crystalline form is characterized by an XRPD pattern comprising 2θ-reflections (±0.2 degrees 2θ) at 8.0°, 16.1°, and 32.5°.

43. (canceled)

44. The crystalline form of claim 42, wherein the XRPD pattern comprises 2θ-reflections (±0.2 degrees 2θ) at 8.0°, 15.2°, 16.1°, 21.5°, 27.6°, and 32.5°.

45. (canceled)

46. The crystalline form of claim 42, wherein the XRPD pattern comprises degree 2θ-reflections (±0.2 degrees 2θ) at 8.0°, 15.2°, 16.1°, 18.6°, 21.5°, 26.1°, 27.6°, 32.5°, and 33.0°.

47. The crystalline form of claim 42, wherein the XRPD pattern is substantially as shown in FIG. 10.

48. A pharmaceutical formulation comprising (a) the salt of claim 1 and (b) a pharmaceutically acceptable excipient.

49.-52. (canceled)

53. A method of treating or preventing a viral infection in a human in need thereof, wherein the method comprises administering to the human the salt of claim 1.

54.-87. (canceled)

88. A method of making a salt of a compound of Formula I: 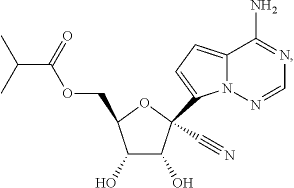 [SMILES: CC(C)C(=O)OC[C@H]1O[C@@](C#N)(c2ccc3c(N)ncnn23)[C@H](O)[C@@H]1O]
comprising treating the compound of Formula I with an acid, wherein the salt is a maleate, oxalate, methanesulfonate, or ethanesulfonate; and wherein the acid is maleic acid, oxalic acid, methanesulfonic acid, or ethanesulfonic acid.

89.-101. (canceled)
